(19) United States
(12) Patent Application Publication
(10) Pub. No.: US 2024/0124564 A1
(43) Pub. Date: Apr. 18, 2024

(54) ANTI-CLEAVED HISTONE H3 MONOCLONAL ANTIBODY THAT SPECIFICALLY RECOGNIZES NEUTROPHIL EXTRACELLULAR TRAPS

(71) Applicant: Max-Planck-Gesellschaft zur Förderung der Wissenschaften e.V., Munich (DE)

(72) Inventors: Dorothea Ogmore Tilley, Dublin (IE); Arturo Zychlinsky, Berlin (DE); Alf Herzig, Berlin (DE)

(21) Appl. No.: 18/278,188

(22) PCT Filed: Feb. 17, 2022
(86) PCT No.: PCT/EP2022/054022

§ 371 Date: Aug. 22, 2023

(30) Foreign Application Priority Data
Feb. 26, 2021 (EP) 21159757.0

(51) Int. Cl. C07K 16/18; G01N 33/68

(52) U.S. Cl. CPC C07K 16/18; G01N 33/6875; C07K 2317/34

(57) ABSTRACT

The present invention relates to a monoclonal antibody 3D9 specifically binding a C-terminal fragment of cleaved histone H3 in NETs that can be used to specifically detect NETs distinguishing them from chromatin of different origin. The invention also provides a method for in vitro detection of neutrophil extracellular traps in isolated biological samples as well as a method for assessing a disease condition associated with NET formation. The present invention also relates to an isolated fragment of human histone H3 cleaved at site L48R49, and to the use of cleavage site L48R49 for specific detection of human neutrophil extracellular traps. The present invention also relates to recombinant nucleic acid sequences encoding said polypeptides, and host cells comprising the same.

The present invention relates to a monoclonal antibody 3D9 specifically binding a C-terminal fragment of cleaved histone H3 in NETs that can be used to specifically detect NETs distinguishing them from chromatin of different origin. The invention also provides a method for in vitro detection of neutrophil extracellular traps in isolated biological samples as well as a method for assessing a disease condition associated with NET formation. The present invention also relates to an isolated purified fragment of human histone H3 cleaved at site L48R49, and to the use of cleavage site L48R49 for specific detection of human neutrophil extracellular traps. The present invention also relates to recombinant nucleic acid sequences encoding said polypeptides, and host cells comprising the same.

### BACKGROUND OF THE INVENTION

Neutrophil extracellular traps (NETs) are histologically defined as areas of decondensed DNA and histones that colocalise with neutrophil granular or cytoplasmic proteins.

NETosis is a regulated form of neutrophil cell death that contributes to the host defense against pathogens and was linked to various diseases soon after its first description in 2004. During NETosis, the neutrophil expels its chromatin, which is decorated with antimicrobial proteins, resulting in a sticky NET that can catch and kill pathogens, preventing them from spreading. This self-sacrificing strategy has initiated a research field that has advanced in the direction of autoimmunity, since defective clearance of NETs can result in an immune response against DNA, histones, and other intracellular proteins that are associated with NETs.5 Neutrophil extracellular traps (NETs) play a role not only in host defense but also in many non-infectious diseases including cancer, thrombosis, autoimmunity and neurological disease.

Currently, NETs are detected by a combination of anti-neutrophil proteins, such as myeloperoxidase (MPO) or neutrophil elastase (NE), and anti-chromatin antibodies as well as DNA stains. Immunofluorescent microscopy is a useful method to detect NETs in tissue sections and in vitro experiments. However, this can be challenging since NET components are distributed across the large decondensed structure resulting in a weak signal. For example, the signal of antibodies to neutrophil elastase (NE) is significantly dimmer in the NETs than in granules of resting cells where this protease is highly concentrated. Conversely, anti-histone antibodies stain NETs strongly but not nuclei of naïve neutrophils, where the chromatin is compact and less accessible. This differential histone staining property is currently exploited for the detection and quantification of NETs (Brinkmann et al., 2012). However, sample preparation and the subsequent image analysis make results between different labs difficult to compare. Thus, there is a need to identify antibodies against NET antigens.

NETs can also be detected on the basis of post-translational modifications (PTMs) that occur during NETosis. One of these PTMs is histone 3 (H3) citrullination by protein arginine deiminase 4 (PAD4) converting arginine residues to citrulline, an amino-acid that is not incorporated into proteins as they are synthesized. Citrullinated H3 (H3cit) is widely used as a surrogate marker of NETs in both in vitro and in vivo experiments. It is important to note that citrullination is not specific to NET formation.

A further PTM occurring during and contributing to NETosis is the cleavage of histones, also named clipping, by granule derived neutrophil proteases. Histone cleavage by cysteine or serine proteases is a bona fide histone PTM that facilitates the gross removal of multiple, subtler, PTMs in the histone tail and is conserved from yeast to mammals.

The study of the role of NETs in disease and as therapeutic targets is hampered by the lack of reliable and specific methods for their detection. There is no available antibody that specifically labels NETs. A NET-specific antibody would be useful for: (1) researchers to identify NETs and (2) for diagnosis—to determine whether complex tissues contain NETs.

It is the objective of the present invention to provide a compound, more specifically an antibody that specifically recognizes Neutrophil Extracellular Traps (NETs). This antibody also facilitates easier NET quantification.

The objective of the present invention is solved by the teaching of the independent claims. Further advantageous features, aspects and details of the invention are evident from the dependent claims, the description, the figures, and the examples of the present application.

### BRIEF DESCRIPTION OF THE INVENTION

In the search for specific markers of NETs, the inventors have identified a unique histone H3 cleavage event occurring in human NET formation, which is positioned at L48R49 of human H3 SEQ ID NO 25 (FIG. 1, FIG. 2).

The inventors exploited the specificity of this event to produce a mouse monoclonal antibody 3D9 (FIG. 3) to the de novo histone H3 epitope positioned at the N-terminal end beginning at R49 of the C-terminal fragment of the cleaved histone H3. In other words, the antibody specifically binds a C-terminal fragment of cleaved histone H3.

By microscopy, this antibody distinguishes NETs from chromatin in purified (FIG. 5) and mixed cell samples (FIG. 6) facilitating simple NET detection and quantification in research. Importantly, 3D9, recognises human NETs induced by both microbial and host derived physiological stimuli (FIG. 5). In addition, this antibody also detects NETs in human tissue sections (FIGS. 8-10). The antibody can also be used for NET quantification through semi-automatic and automatic methods (FIG. 4). Importantly, the antibody 3D9 can distinguish between NETosis and other form of cell death in neutrophils (FIG. 7A), whereas the anti-chromatin antibody PL2.3 does not (FIG. 7B).

Thus, the inventors have demonstrated that this antibody is a new tool to detect and quantify NETs, facilitating comparison of results between laboratories and a tool to examine biopsies and other complex tissues for NETs.

The antibody of the present invention recognizes a de novo epitope positioned at position R49 in histone H3 that appears only during NET formation. Hence, this antibody is the first to recognize NETs specifically.

Until now, histone citrullination H3cit is the only family of PTMs that have been used for antibody-based detection of NETs. Herein the inventors have shown histone cleavage at H3R49 as a new histone PTM that can be used for broad detection of NETs from human samples. The use of H3cit for the detection of NETs is not without controversy. Not all NETs are citrullinated and NET formation can occur in the absence or inhibition of citrullinating enzymes. By identifying the precise histone cleavage site, the inventors shed further light on this. The most commonly used H3cit antibody detects citrullination of R2, R8 and R17. However, histone cleavage at R49 would remove the H3cit epitopes, rendering these NET defining PTMs mutually exclusive on a single histone level. Indeed, co-staining by anti-H3cit and 3D9 in inflamed kidney (FIG. 8C), gall bladder (FIG. 9) and appendix (FIG. 10) paraffin sections revealed extensive mutual exclusion of the two marks and more abundant staining of decondensed chromatin by 3D9. Thus, the inventors have shown that 3D9 will allow broad detection of NETs but may display a preference for more mature or proteolytically processed NETs.

In contrast to the present study, histone cleavage was recently reported as discriminating between different pathways of NET formation (Pieterse et al., 2018, Annals of the rheumatic diseases 77, 1790-1798). Using a sandwich ELISA approach, Pieterse et al. concluded that, generally, the N-terminal histone tails are removed in NOX dependent but not NOX independent NET formation. They used a suite of N-terminal directed antibodies for H2B, H3 and H4, and all H3 epitopes were located N-terminal to the cleavage site H3R49. However, in the final biological sample testing the authors did not examine H3. Interestingly, the authors observed that, by immunofluorescent microscopy, all histone N-terminal antibodies failed to stain NETs at time points after cell lysis.

The inventors detected NETs in fixed or denatured human samples from in vitro experiments and histological samples.

Thus, histone cleavage at H3R49 is a novel and so far undescribed cleavage site in any eukaryotic organism.

Unusually, it is located in the globular rather than the unstructured tail region of H3.

Thus, the present invention relates to an isolated antibody specifically binding a C-terminal fragment of cleaved histone H3 or an antigen-binding portion thereof, wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8.

The present invention is also directed to an isolated antibody specifically binding a C-terminal fragment of cleaved histone H3 or an antigen-binding portion thereof as described above, wherein the antibody comprises:
I) a heavy chain variable region comprising an amino acid sequence of SEQ ID NO: 1, or an amino acid sequence having a sequence identity of more than 90% with of SEQ ID NO: 1; and
II) a light chain variable region comprising an amino acid sequence of SEQ ID NO: 2, or an amino acid sequence having a sequence identity of more than 90% with of SEQ ID NO: 2.

A preferred embodiment of the invention is directed to an isolated antibody specifically binding a C-terminal fragment of cleaved histone H3 or an antigen-binding portion thereof as described above, wherein the antigen binding fragment is selected from the group comprising single chain variable fragments (scFv), variable domain (Fv) fragments, fragment antigen binding (Fab) fragments, F(ab)2 fragments, peptides, or proteolytic fragments containing an epitope binding region.

Another preferred embodiment of the invention is directed to a nucleic acid composition comprising one or more nucleic acid molecules encoding an antibody specifically binding a C-terminal fragment of cleaved histone H3 or an antigen-binding portion thereof as described above.

A more preferred embodiment of the invention is directed to a nucleic acid composition comprising one or more nucleic acid molecules encoding an antibody specifically binding a C-terminal fragment of cleaved histone H3 or an antigen-binding portion thereof as described above, wherein the nucleic acid composition comprises a nucleic acid sequence having a sequence identity of more than 90% with SEQ ID NO: 9, and a nucleic acid sequence having a sequence identity of more than 90% with SEQ ID NO: 10.

A further preferred embodiment of the invention is directed to a host cell comprising the recombinant nucleic acid molecule as described above.

Another embodiment of the invention provides a method for in vitro detection of neutrophil extracellular traps in an isolated biological sample, the method comprising:
1) contacting said isolated biological sample with an antibody specific for a C-terminal fragment of cleaved histone H3 or an antigen-binding portion thereof as described above,
2) providing conditions sufficient to allow said antibody specific for a C-terminal fragment of cleaved histone H3 or an antigen-binding portion thereof to bind to said isolated biological sample,
3) determining whether the antibody specific for a C-terminal fragment of cleaved histone H3 or an antigen-binding portion thereof binds to said isolated biological sample.

A further embodiment of the invention provides a method as described above, wherein the antibody specific for a C-terminal fragment of cleaved histone H3 or an antigen-binding portion thereof is linked to a detectable label.

A preferred embodiment of the invention provides a method for in vitro assessing a disease condition in an individual:
i) contacting an isolated biological sample obtained from said individual with an antibody specific for a C-terminal fragment of cleaved histone H3 or an antigen-binding portion thereof as described above;
ii) providing conditions sufficient to allow said antibody specific for a C-terminal fragment of cleaved histone H3 or an antigen-binding portion thereof to bind to said isolated biological sample,
iii) determining whether the antibody specific for a C-terminal fragment of cleaved histone H3 or an antigen-binding portion thereof binds to said isolated biological sample,
   wherein said disease is associated with NET formation,
   wherein said disease is selected from the group comprising thrombotic disease, neurological disease, bacterial infection disease, viral infection disease, fungal infection disease, inflammatory disease, autoimmune disease, cancer disease, cancer metastasis disease.

Another preferred embodiment of the invention provides a method as described above, wherein the autoimmune disease is selected from the group comprising psoriasis; vasculitis; systemic lupus erythematosus (SLE); rheumatoid arthritis; ulcerative colitis; Crohn's disease; immune-mediated or Type 1 Diabetes Mellitus; immune mediated glomerulonephritis; inflammatory bowel diseases (IBD); antiphospholipid antibody syndrome; immune thrombocytopenia.

Another preferred embodiment of the invention provides a method as described above, wherein the antibody specific for a C-terminal fragment of cleaved histone H3 or an antigen-binding portion thereof is linked to a detectable label.

A further preferred embodiment of the invention provides an isolated fragment of human histone H3 cleaved at site L48R49 of SEQ ID NO 25, wherein the fragment of human histone H3 cleaved at site L48R49 consists of an amino acid sequence comprised between residues R49-A135 of SEQ ID NO 25, or a segment thereof, or consists of an amino acid sequence comprised between residues A1-L48 of SEQ ID NO 25, or a segment thereof.

In another aspect, the present invention provides a kit for assessing a disease containing the antibody specific for a C-terminal fragment or an antigen binding portion thereof as described above, and/or the fragment of human histone H3 as described above, wherein the disease is selected from the group comprising thrombotic disease, neurological disease, bacterial infection disease, viral infection disease, fungal infection disease, inflammatory disease, autoimmune disease, cancer disease, cancer metastasis disease.

In a further aspect, the present invention provides a kit as described above, wherein the autoimmune disease is selected from the group comprising psoriasis; vasculitis; systemic lupus erythematosus (SLE); rheumatoid arthritis; ulcerative colitis; Crohn's disease; immune-mediated or Type 1 Diabetes Mellitus; immune mediated glomerulonephritis; inflammatory bowel diseases (IBD); antiphospholipid antibody syndrome; immune thrombocytopenia.

In a particular aspect, the present invention provides a use of cleavage site of histone H3 situated at L48R49 of SEQ ID NO 25 for specific detection of neutrophil extracellular traps.

### DESCRIPTION OF THE INVENTION

The present invention relates to an isolated antibody specifically binding a C-terminal fragment of cleaved histone H3 or an antigen-binding portion thereof, wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8.

### Definitions

An “antibody specifically binding a C-terminal fragment of cleaved histone H3” refers to an antibody that specifically binds to the de novo histone H3 epitope positioned at the N-terminal end beginning at R49 of the C-terminal fragment of the histone H3 cleaved at site L48R49 on SEQ ID NO 25.

“L48R49” and “H3R49” are used herein interchangeably to indicate the cleavage site when referring to SEQ ID NO 25.

SEQ ID NO 25 corresponds to histone H3 sequence Q71 D13 without the first Met. Thus, the cleavage site L48R49 on SEQ ID NO 25 corresponds to the cleavage site L49R50 on Q71 DI3, and on the other H3 variant sequences P0DPK2, Q6NXT2, Q16695, P84243, and P68431 (Example 9).

Thus, the cleavage site L48R49 refers also to the cleavage site present in other variants or homologues of histone H3 which comprise a sequence having at least 90% identity with SEQ ID NO 25.

The cleavage at site L48R49 itself occurs closer to the N terminal domain of histone H3. This results in 2 fragments, one of which is called “N terminal fragment” of histone H3. The other larger fragment is composed of the unstructured “C terminal domain” and the main “helical domain” of histone H3. The antigen epitope is located at the N terminal end of this helical domain, also referred to herein as “C-terminal fragment” of histone H3.

Thus, the present invention also relates to an isolated antibody specifically binding a C-terminal fragment of histone H3 cleaved at L48R49 of SEQ ID NO 25, or an antigen-binding portion thereof, wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8.

The present invention is also directed to an isolated antibody specifically binding a C-terminal fragment of cleaved histone H3 or an antigen-binding portion thereof, wherein the antibody comprises:
I) a heavy chain variable region comprising an amino acid sequence of SEQ ID NO: 1, or an amino acid sequence having a sequence identity of more than 90% with of SEQ ID NO: 1; and
II) a light chain variable region comprising an amino acid sequence of SEQ ID NO: 2, or an amino acid sequence having a sequence identity of more than 90% with of SEQ ID NO: 2.

The present invention is further directed to an isolated antibody specifically binding a C-terminal fragment of histone H3 cleaved at L48R49 of SEQ ID NO 25, or an antigen-binding portion thereof, wherein the antibody comprises:
I) a heavy chain variable region comprising an amino acid sequence of SEQ ID NO: 1, or an amino acid sequence having a sequence identity of more than 90% with of SEQ ID NO: 1; and
II) a light chain variable region comprising an amino acid sequence of SEQ ID NO: 2, or an amino acid sequence having a sequence identity of more than 90% with of SEQ ID NO: 2.

Thus, the present invention also relates to an isolated antibody specifically binding a C-terminal fragment of cleaved histone H3, or an antigen-binding portion thereof, wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antibody comprises:
I) a heavy chain variable region comprising an amino acid sequence of SEQ ID NO: 1, or an amino acid sequence having a sequence identity of more than 90% with of SEQ ID NO: 1; and
II) a light chain variable region comprising an amino acid sequence of SEQ ID NO: 2, or an amino acid sequence having a sequence identity of more than 90% with of SEQ ID NO: 2.

A preferred embodiment of the invention is directed to an isolated antibody specifically binding a C-terminal fragment of cleaved histone H3 or an antigen-binding portion as described above, wherein the antigen binding fragment is selected from the group comprising single chain variable fragments (scFv), variable domain (Fv) fragments, fragment antigen binding (Fab) fragments, F(ab)2 fragments, peptides, or proteolytic fragments containing an epitope binding region.

A preferred embodiment of the invention is directed to an isolated antibody specifically binding a C-terminal fragment of cleaved histone H3, or an antigen-binding portion thereof, wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antigen binding fragment is selected from the group comprising single chain variable fragments (scFv), variable domain (Fv) fragments, fragment antigen binding (Fab) fragments, F(ab)2 fragments, peptides, or proteolytic fragments containing an epitope binding region.

A further preferred embodiment of the invention is directed to an isolated antibody specifically binding a C-terminal fragment of cleaved histone H3, or an antigen-binding portion thereof, wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antibody comprises:
I) a heavy chain variable region comprising an amino acid sequence of SEQ ID NO: 1, or an amino acid sequence having a sequence identity of more than 90% with of SEQ ID NO: 1; and
II) a light chain variable region comprising an amino acid sequence of SEQ ID NO: 2, or an amino acid sequence having a sequence identity of more than 90% with of SEQ ID NO: 2,

wherein the antigen binding fragment is selected from the group comprising single chain variable fragments (scFv), variable domain (Fv) fragments, fragment antigen binding (Fab) fragments, F(ab)2 fragments, peptides, or proteolytic fragments containing an epitope binding region.

A further more preferred embodiment of the invention is directed to an isolated antibody specifically binding a C-terminal fragment of cleaved histone H3, or an antigen-binding portion thereof, wherein the antibody comprises:
I) a heavy chain variable region comprising an amino acid sequence of SEQ ID NO: 1, or an amino acid sequence having a sequence identity of more than 90% with of SEQ ID NO: 1; and
II) a light chain variable region comprising an amino acid sequence of SEQ ID NO: 2, or an amino acid sequence having a sequence identity of more than 90% with of SEQ ID NO: 2,

wherein the antigen binding fragment is selected from the group comprising single chain variable fragments (scFv), variable domain (Fv) fragments, fragment antigen binding (Fab) fragments, F(ab)2 fragments, peptides, or proteolytic fragments containing an epitope binding region.

A preferred embodiment of the invention is directed to an isolated antibody specifically binding a C-terminal fragment of histone H3 cleaved at L48R49 of SEQ ID NO 25, or an antigen-binding portion thereof, wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antigen binding fragment is selected from the group comprising single chain variable fragments (scFv), variable domain (Fv) fragments, fragment antigen binding (Fab) fragments, F(ab)2 fragments, peptides, or proteolytic fragments containing an epitope binding region.

A further preferred embodiment of the invention is directed to an isolated antibody specifically binding a C-terminal fragment of histone H3 cleaved at L48R49 of SEQ ID NO 25, or an antigen-binding portion thereof, wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antibody comprises:
I) a heavy chain variable region comprising an amino acid sequence of SEQ ID NO: 1, or an amino acid sequence having a sequence identity of more than 90% with of SEQ ID NO: 1; and
II) a light chain variable region comprising an amino acid sequence of SEQ ID NO: 2, or an amino acid sequence having a sequence identity of more than 90% with of SEQ ID NO: 2,

wherein the antigen binding fragment is selected from the group comprising single chain variable fragments (scFv), variable domain (Fv) fragments, fragment antigen binding (Fab) fragments, F(ab)2 fragments, peptides, or proteolytic fragments containing an epitope binding region.

A further more preferred embodiment of the invention is directed to an isolated antibody specifically binding a C-terminal fragment of histone H3 cleaved at L48R49 of SEQ ID NO 25, or an antigen-binding portion thereof, wherein the antibody comprises:
I) a heavy chain variable region comprising an amino acid sequence of SEQ ID NO: 1, or an amino acid sequence having a sequence identity of more than 90% with of SEQ ID NO: 1; and
II) a light chain variable region comprising an amino acid sequence of SEQ ID NO: 2, or an amino acid sequence having a sequence identity of more than 90% with of SEQ ID NO: 2,

wherein the antigen binding fragment is selected from the group comprising single chain variable fragments (scFv), variable domain (Fv) fragments, fragment antigen binding (Fab) fragments, F(ab)2 fragments, peptides, or proteolytic fragments containing an epitope binding region.

Nucleic Acids

A preferred embodiment of the invention is directed to a nucleic acid composition comprising one or more nucleic acid molecules encoding an antibody specifically binding a C-terminal fragment of cleaved histone H3 or an antigen-binding portion thereof, wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8.

Another preferred embodiment of the invention is directed to a nucleic acid composition comprising one or more nucleic acid molecules encoding an antibody specifically binding a C-terminal fragment of cleaved histone H3 or an antigen-binding portion thereof, wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and wherein the antibody comprises:
I) a heavy chain variable region comprising an amino acid sequence of SEQ ID NO: 1, or an amino acid sequence having a sequence identity of more than 90% with of SEQ ID NO: 1; and
II) a light chain variable region comprising an amino acid sequence of SEQ ID NO: 2, or an amino acid sequence having a sequence identity of more than 90% with of SEQ ID NO: 2.

Another further preferred embodiment of the invention is directed to a nucleic acid composition comprising one or more nucleic acid molecules encoding an antibody specifically binding a C-terminal fragment of cleaved histone H3 or an antigen-binding portion thereof, wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antigen binding fragment is selected from the group comprising single chain variable fragments (scFv), variable domain (Fv) fragments, fragment antigen binding (Fab) fragments, F(ab)2 fragments, peptides, or proteolytic fragments containing an epitope binding region.

Another further preferred embodiment of the invention is directed to a nucleic acid composition comprising one or more nucleic acid molecules encoding an antibody specifically binding a C-terminal fragment of cleaved histone H3 or an antigen-binding portion thereof, wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antibody comprises:
I) a heavy chain variable region comprising an amino acid sequence of SEQ ID NO: 1, or an amino acid sequence having a sequence identity of more than 90% with of SEQ ID NO: 1; and
II) a light chain variable region comprising an amino acid sequence of SEQ ID NO: 2, or an amino acid sequence having a sequence identity of more than 90% with of SEQ ID NO: 2, and

wherein the antigen binding fragment is selected from the group comprising single chain variable fragments (scFv), variable domain (Fv) fragments, fragment antigen binding (Fab) fragments, F(ab)2 fragments, peptides, or proteolytic fragments containing an epitope binding region.

More preferably, the nucleic acid composition comprises a nucleic acid sequence having a sequence identity of more than 90% with SEQ ID NO: 9, and a nucleic acid sequence having a sequence identity of more than 90% with SEQ ID NO: 10.

A preferred embodiment of the invention is directed to a nucleic acid composition comprising one or more nucleic acid molecules encoding an antibody specifically binding a C-terminal fragment of cleaved histone H3 or an antigen-binding portion thereof, wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the nucleic acid composition comprises a nucleic acid sequence having a sequence identity of more than 90% with SEQ ID NO: 9, and a nucleic acid sequence having a sequence identity of more than 90% with SEQ ID NO: 10.

Another preferred embodiment of the invention is directed to a nucleic acid composition comprising one or more nucleic acid molecules encoding an antibody specifically binding a C-terminal fragment of cleaved histone H3 or an antigen-binding portion thereof, wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antibody comprises:
I) a heavy chain variable region comprising an amino acid sequence of SEQ ID NO: 1, or an amino acid sequence having a sequence identity of more than 90% with of SEQ ID NO: 1; and
II) a light chain variable region comprising an amino acid sequence of SEQ ID NO: 2, or an amino acid sequence having a sequence identity of more than 90% with of SEQ ID NO: 2; and

wherein the nucleic acid composition comprises a nucleic acid sequence having a sequence identity of more than 90% with SEQ ID NO: 9, and a nucleic acid sequence having a sequence identity of more than 90% with SEQ ID NO: 10.

Another further preferred embodiment of the invention is directed to a nucleic acid composition comprising one or more nucleic acid molecules encoding an antibody specifically binding a C-terminal fragment of cleaved histone H3 or an antigen-binding portion thereof, wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antigen binding fragment is selected from the group comprising single chain variable fragments (scFv), variable domain (Fv) fragments, fragment antigen binding (Fab) fragments, F(ab)2 fragments, peptides, or proteolytic fragments containing an epitope binding region; and

wherein the nucleic acid composition comprises a nucleic acid sequence having a sequence identity of more than 90% with SEQ ID NO: 9, and a nucleic acid sequence having a sequence identity of more than 90% with SEQ ID NO: 10.

Another further preferred embodiment of the invention is directed to a nucleic acid composition comprising one or more nucleic acid molecules encoding an antibody specifically binding a C-terminal fragment of cleaved histone H3 or an antigen-binding portion thereof, wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antibody comprises:
I) a heavy chain variable region comprising an amino acid sequence of SEQ ID NO: 1, or an amino acid sequence having a sequence identity of more than 90% with of SEQ ID NO: 1; and
II) a light chain variable region comprising an amino acid sequence of SEQ ID NO: 2, or an amino acid sequence having a sequence identity of more than 90% with of SEQ ID NO: 2, and

wherein the antigen binding fragment is selected from the group comprising single chain variable fragments (scFv), variable domain (Fv) fragments, fragment antigen binding (Fab) fragments, F(ab)2 fragments, peptides, or proteolytic fragments containing an epitope binding region; and

wherein the nucleic acid composition comprises a nucleic acid sequence having a sequence identity of more than 90% with SEQ ID NO: 9, and a nucleic acid sequence having a sequence identity of more than 90% with SEQ ID NO: 10.

Host Cell

The present invention also provides a host cell comprising a recombinant nucleic acid molecule for coding an antibody specifically binding a C-terminal fragment of cleaved histone H3 or an antigen-binding portion thereof.

Thus, another embodiment of the invention is directed to a host cell comprising a nucleic acid composition comprising one or more nucleic acid molecules encoding an antibody specifically binding a C-terminal fragment of cleaved histone H3 or an antigen-binding portion thereof, wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8.

Another preferred embodiment of the invention is directed to a host cell comprising a nucleic acid composition comprising one or more nucleic acid molecules encoding an antibody specifically binding a C-terminal fragment of cleaved histone H3 or an antigen-binding portion thereof, wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antibody comprises:
I) a heavy chain variable region comprising an amino acid sequence of SEQ ID NO: 1, or an amino acid sequence having a sequence identity of more than 90% with of SEQ ID NO: 1; and
II) a light chain variable region comprising an amino acid sequence of SEQ ID NO: 2, or an amino acid sequence having a sequence identity of more than 90% with of SEQ ID NO: 2.

Another further preferred embodiment of the invention is directed to a host cell comprising a nucleic acid composition comprising one or more nucleic acid molecules encoding an antibody specifically binding a C-terminal fragment of cleaved histone H3 or an antigen-binding portion thereof, wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and
wherein the antigen binding fragment is selected from the group comprising single chain variable fragments (scFv), variable domain (Fv) fragments, fragment antigen binding (Fab) fragments, F(ab)2 fragments, peptides, or proteolytic fragments containing an epitope binding region.

Another further preferred embodiment of the invention is directed to a host cell comprising a nucleic acid composition comprising one or more nucleic acid molecules encoding an antibody specifically binding a C-terminal fragment of cleaved histone H3 or an antigen-binding portion thereof, wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antibody comprises:
I) a heavy chain variable region comprising an amino acid sequence of SEQ ID NO: 1, or an amino acid sequence having a sequence identity of more than 90% with of SEQ ID NO: 1; and
II) a light chain variable region comprising an amino acid sequence of SEQ ID NO: 2, or an amino acid sequence having a sequence identity of more than 90% with of SEQ ID NO: 2, and

wherein the antigen binding fragment is selected from the group comprising single chain variable fragments (scFv), variable domain (Fv) fragments, fragment antigen binding (Fab) fragments, F(ab)2 fragments, peptides, or proteolytic fragments containing an epitope binding region.

Therefore, an embodiment of the invention is directed to a host cell comprising a nucleic acid composition comprising one or more nucleic acid molecules encoding an antibody specifically binding a C-terminal fragment of cleaved histone H3 or an antigen-binding portion thereof, wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the nucleic acid composition comprises a nucleic acid sequence having a sequence identity of more than 90% with SEQ ID NO: 9, and a nucleic acid sequence having a sequence identity of more than 90% with SEQ ID NO: 10.

Another embodiment of the invention is directed to a host cell comprising a nucleic acid composition comprising one or more nucleic acid molecules encoding an antibody specifically binding a C-terminal fragment of cleaved histone H3 or an antigen-binding portion thereof, wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antibody comprises:
I) a heavy chain variable region comprising an amino acid sequence of SEQ ID NO: 1, or an amino acid sequence having a sequence identity of more than 90% with of SEQ ID NO: 1; and
II) a light chain variable region comprising an amino acid sequence of SEQ ID NO: 2, or an amino acid sequence having a sequence identity of more than 90% with of SEQ ID NO: 2; and

wherein the nucleic acid composition comprises a nucleic acid sequence having a sequence identity of more than 90% with SEQ ID NO: 9, and a nucleic acid sequence having a sequence identity of more than 90% with SEQ ID NO: 10.

Another further preferred embodiment of the invention is directed to a host cell comprising a nucleic acid composition comprising one or more nucleic acid molecules encoding an antibody specifically binding a C-terminal fragment of cleaved histone H3 or an antigen-binding portion thereof, wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antigen binding fragment is selected from the group comprising single chain variable fragments (scFv), variable domain (Fv) fragments, fragment antigen binding (Fab) fragments, F(ab)2 fragments, peptides, or proteolytic fragments containing an epitope binding region; and

wherein the nucleic acid composition comprises a nucleic acid sequence having a sequence identity of more than 90% with SEQ ID NO: 9, and a nucleic acid sequence having a sequence identity of more than 90% with SEQ ID NO: 10.

Another further preferred embodiment of the invention is directed to a host cell comprising a nucleic acid composition comprising one or more nucleic acid molecules encoding an antibody specifically binding a C-terminal fragment of cleaved histone H3 or an antigen-binding portion thereof, wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antibody comprises:
I) a heavy chain variable region comprising an amino acid sequence of SEQ ID NO: 1, or an amino acid sequence having a sequence identity of more than 90% with of SEQ ID NO: 1; and
II) a light chain variable region comprising an amino acid sequence of SEQ ID NO: 2, or an amino acid sequence having a sequence identity of more than 90% with of SEQ ID NO: 2, and

wherein the antigen binding fragment is selected from the group comprising single chain variable fragments (scFv), variable domain (Fv) fragments, fragment antigen binding (Fab) fragments, F(ab)2 fragments, peptides, or proteolytic fragments containing an epitope binding region; and

wherein the nucleic acid composition comprises a nucleic acid sequence having a sequence identity of more than 90% with SEQ ID NO: 9, and a nucleic acid sequence having a sequence identity of more than 90% with SEQ ID NO: 10.

Method for in vitro detection of NETs Another embodiment of the invention provides a method for in vitro detection of neutrophil extracellular traps in an isolated biological sample, the method comprising:
1) contacting said isolated biological sample with an antibody specific for a C-terminal fragment of cleaved histone H3 or an antigen-binding portion thereof,
2) providing conditions sufficient to allow said antibody specific for a C-terminal fragment of cleaved histone H3 or an antigen-binding portion thereof to bind to said isolated biological sample,
3) determining whether the antibody specific for a C-terminal fragment of cleaved histone H3 or an antigen-binding portion thereof binds to said isolated biological sample, and

wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8.

A further embodiment of the invention provides a method for in vitro detection of neutrophil extracellular traps in an isolated biological sample, the method comprising:
1) contacting said isolated biological sample with an antibody specific for a C-terminal fragment of cleaved histone H3 or an antigen-binding portion thereof,
2) providing conditions sufficient to allow said antibody specific for a C-terminal fragment of cleaved histone H3 or an antigen-binding portion thereof to bind to said isolated biological sample,
3) determining whether the antibody specific for a C-terminal fragment of cleaved histone H3 or an antigen-binding portion thereof binds to said isolated biological sample, and

wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antibody comprises:
I) a heavy chain variable region comprising an amino acid sequence of SEQ ID NO: 1, or an amino acid sequence having a sequence identity of more than 90% with of SEQ ID NO: 1; and
II) a light chain variable region comprising an amino acid sequence of SEQ ID NO: 2, or an amino acid sequence having a sequence identity of more than 90% with of SEQ ID NO: 2.

Another preferred embodiment of the invention provides a method for in vitro detection of neutrophil extracellular traps in an isolated biological sample, the method comprising:
1) contacting said isolated biological sample with an antibody specific for a C-terminal fragment of cleaved histone H3 or an antigen-binding portion thereof,
2) providing conditions sufficient to allow said antibody specific for a C-terminal fragment of cleaved histone H3 or an antigen-binding portion thereof to bind to said isolated biological sample,
3) determining whether the antibody specific for a C-terminal fragment of cleaved histone H3 or an antigen-binding portion thereof binds to said isolated biological sample, and

wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antigen binding fragment is selected from the group comprising single chain variable fragments (scFv), variable domain (Fv) fragments, fragment antigen binding (Fab) fragments, F(ab)2 fragments, peptides, or proteolytic fragments containing an epitope binding region.

Another more preferred embodiment of the invention provides a method for in vitro detection of neutrophil extracellular traps in an isolated biological sample, the method comprising:
1) contacting said isolated biological sample with an antibody specific for a C-terminal fragment of cleaved histone H3 or an antigen-binding portion thereof,
2) providing conditions sufficient to allow said antibody specific for a C-terminal fragment of cleaved histone H3 or an antigen-binding portion thereof to bind to said isolated biological sample,
3) determining whether the antibody specific for a C-terminal fragment of cleaved histone H3 or an antigen-binding portion thereof binds to said isolated biological sample, and

wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antibody comprises:
I) a heavy chain variable region comprising an amino acid sequence of SEQ ID NO: 1, or an amino acid sequence having a sequence identity of more than 90% with of SEQ ID NO: 1; and
II) a light chain variable region comprising an amino acid sequence of SEQ ID NO: 2, or an amino acid sequence having a sequence identity of more than 90% with of SEQ ID NO: 2, and

wherein the antigen binding fragment is selected from the group comprising single chain variable fragments (scFv), variable domain (Fv) fragments, fragment antigen binding (Fab) fragments, F(ab)2 fragments, peptides, or proteolytic fragments containing an epitope binding region.

In some embodiments is preferred when the antibody specific for a C-terminal fragment of cleaved histone H3 or an antigen-binding portion is linked to a detectable label.

Thus, another embodiment of the invention provides a method for in vitro detection of neutrophil extracellular traps in an isolated biological sample, the method comprising:
1) contacting said isolated biological sample with an antibody specific for a C-terminal fragment of cleaved histone H3 or an antigen-binding portion thereof,
2) providing conditions sufficient to allow said antibody specific for a C-terminal fragment of cleaved histone H3 or an antigen-binding portion thereof to bind to said isolated biological sample,
3) determining whether the antibody specific for a C-terminal fragment of cleaved histone H3 or an antigen-binding portion thereof binds to said isolated biological sample, and

wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antibody specific for a C-terminal fragment of cleaved histone H3 or an antigen-binding portion is linked to a detectable label.

A further embodiment of the invention provides a method for in vitro detection of neutrophil extracellular traps in an isolated biological sample, the method comprising:
1) contacting said isolated biological sample with an antibody specific for a C-terminal fragment of cleaved histone H3 or an antigen-binding portion thereof,
2) providing conditions sufficient to allow said antibody specific for a C-terminal fragment of cleaved histone H3 or an antigen-binding portion thereof to bind to said isolated biological sample,
3) determining whether the antibody specific for a C-terminal fragment of cleaved histone H3 or an antigen-binding portion thereof binds to said isolated biological sample, and

wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antibody comprises:
I) a heavy chain variable region comprising an amino acid sequence of SEQ ID NO: 1, or an amino acid sequence having a sequence identity of more than 90% with of SEQ ID NO: 1; and
II) a light chain variable region comprising an amino acid sequence of SEQ ID NO: 2, or an amino acid sequence having a sequence identity of more than 90% with of SEQ ID NO: 2, and

wherein the antibody specific for a C-terminal fragment of cleaved histone H3 or an antigen-binding portion is linked to a detectable label.

Another preferred embodiment of the invention provides a method for in vitro detection of neutrophil extracellular traps in an isolated biological sample, the method comprising:
1) contacting said isolated biological sample with an antibody specific for a C-terminal fragment of cleaved histone H3 or an antigen-binding portion thereof,
2) providing conditions sufficient to allow said antibody specific for a C-terminal fragment of cleaved histone H3 or an antigen-binding portion thereof to bind to said isolated biological sample,
3) determining whether the antibody specific for a C-terminal fragment of cleaved histone H3 or an antigen-binding portion thereof binds to said isolated biological sample, and

wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antigen binding fragment is selected from the group comprising single chain variable fragments (scFv), variable domain (Fv) fragments, fragment antigen binding (Fab) fragments, F(ab)2 fragments, peptides, or proteolytic fragments containing an epitope binding region, and

wherein the antibody specific for a C-terminal fragment of cleaved histone H3 or an antigen-binding portion is linked to a detectable label.

Another more preferred embodiment of the invention provides a method for in vitro detection of neutrophil extracellular traps in an isolated biological sample, the method comprising:
1) contacting said isolated biological sample with an antibody specific for a C-terminal fragment of cleaved histone H3 or an antigen-binding portion thereof,
2) providing conditions sufficient to allow said antibody specific for a C-terminal fragment of cleaved histone H3 or an antigen-binding portion thereof to bind to said isolated biological sample,
3) determining whether the antibody specific for a C-terminal fragment of cleaved histone H3 or an antigen-binding portion thereof binds to said isolated biological sample, and

wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antibody comprises:
I) a heavy chain variable region comprising an amino acid sequence of SEQ ID NO: 1, or an amino acid sequence having a sequence identity of more than 90% with of SEQ ID NO: 1; and
II) a light chain variable region comprising an amino acid sequence of SEQ ID NO: 2, or an amino acid sequence having a sequence identity of more than 90% with of SEQ ID NO: 2, and

wherein the antigen binding fragment is selected from the group comprising single chain variable fragments (scFv), variable domain (Fv) fragments, fragment antigen binding (Fab) fragments, F(ab)2 fragments, peptides, or proteolytic fragments containing an epitope binding region, and

wherein the antibody specific for a C-terminal fragment of cleaved histone H3 or an antigen-binding portion is linked to a detectable label.

The biological sample is preferably selected from a cell sample, mixed cell sample, a cell line sample, a primary cell sample, a blood sample, a peripheral blood mononuclear cell (PBMC) sample, a neutrophil sample, a tissue sample, a histological tissue sample, for example a tissue sample embedded in paraffin or a cryopreserved tissue sample.

Method for in vitro assessing of a disease associated with NET formation.

A preferred embodiment of the invention provides a method for in vitro assessing a disease condition in an individual:
i) contacting an isolated biological sample obtained from said individual with an antibody specific for a C-terminal fragment of cleaved histone H3 or an antigen-binding portion thereof as described above;
ii) providing conditions sufficient to allow said antibody specific for a C-terminal fragment of cleaved histone H3 or an antigen-binding portion thereof to bind to said isolated biological sample,
iii) determining whether the antibody specific for a C-terminal fragment of cleaved histone H3 or an antigen-binding portion thereof binds to said isolated biological sample,
   wherein said disease is associated with NET formation,
   wherein said disease is selected from the group comprising thrombotic disease, neurological disease, bacterial infection disease, viral infection disease, fungal infection disease, inflammatory disease, autoimmune disease, cancer disease, cancer metastasis disease, and

wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8.

The biological sample is preferably selected from a cell sample, mixed cell sample, a cell line sample, a primary cell sample, a blood sample, a peripheral blood mononuclear cell (PBMC) sample, a neutrophil sample, a tissue sample, a histological tissue sample, for example a tissue sample embedded in paraffin or a cryopreserved tissue sample.

Thus, a more preferred embodiment of the invention provides a method for in vitro assessing a disease condition in an individual:
i) contacting an isolated biological sample obtained from said individual with an antibody specific for a C-terminal fragment of cleaved histone H3 or an antigen-binding portion thereof as described above;
ii) providing conditions sufficient to allow said antibody specific for a C-terminal fragment of cleaved histone H3 or an antigen-binding portion thereof to bind to said isolated biological sample,
iii) determining whether the antibody specific for a C-terminal fragment of cleaved histone H3 or an antigen-binding portion thereof binds to said isolated biological sample, wherein said disease is associated with NET formation, wherein said disease is selected from the group comprising thrombotic disease, neurological disease, bacterial infection disease, viral infection disease, fungal infection disease, inflammatory disease, autoimmune disease, cancer disease, cancer metastasis disease, and

wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the biological sample is selected from a cell sample, mixed cell sample, a cell line sample, a primary cell sample, a blood sample, a peripheral blood mononuclear cell (PBMC) sample, a neutrophil sample, a tissue sample, a histological tissue sample, a tissue sample embedded in paraffin or a cryopreserved tissue sample.

A preferred embodiment of the invention provides a method for in vitro assessing a disease condition in an individual:
i) contacting an isolated biological sample obtained from said individual with an antibody specific for a C-terminal fragment of cleaved histone H3 or an antigen-binding portion thereof as described above;
ii) providing conditions sufficient to allow said antibody specific for a C-terminal fragment of cleaved histone H3 or an antigen-binding portion thereof to bind to said isolated biological sample,
iii) determining whether the antibody specific for a C-terminal fragment of cleaved histone H3 or an antigen-binding portion thereof binds to said isolated biological sample,
   wherein said disease is associated with NET formation,
   wherein said disease is selected from the group comprising thrombotic disease, neurological disease, bacterial infection disease, viral infection disease, fungal infection disease, inflammatory disease, autoimmune disease, cancer disease, cancer metastasis disease, and

wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antibody comprises:
I) a heavy chain variable region comprising an amino acid sequence of SEQ ID NO: 1, or an amino acid sequence having a sequence identity of more than 90% with of SEQ ID NO: 1; and
II) a light chain variable region comprising an amino acid sequence of SEQ ID NO: 2, or an amino acid sequence having a sequence identity of more than 90% with of SEQ ID NO: 2.

A more preferred embodiment of the invention provides a method for in vitro assessing a disease condition in an individual:
i) contacting an isolated biological sample obtained from said individual with an antibody specific for a C-terminal fragment of cleaved histone H3 or an antigen-binding portion thereof as described above;
ii) providing conditions sufficient to allow said antibody specific for a C-terminal fragment of cleaved histone H3 or an antigen-binding portion thereof to bind to said isolated biological sample,
iii) determining whether the antibody specific for a C-terminal fragment of cleaved histone H3 or an antigen-binding portion thereof binds to said isolated biological sample,
   wherein said disease is associated with NET formation,
   wherein said disease is selected from the group comprising thrombotic disease, neurological disease, bacterial infection disease, viral infection disease, fungal infection disease, inflammatory disease, autoimmune disease, cancer disease, cancer metastasis disease, and

wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antigen binding fragment is selected from the group comprising single chain variable fragments (scFv), variable domain (Fv) fragments, fragment antigen binding (Fab) fragments, F(ab)2 fragments, peptides, or proteolytic fragments containing an epitope binding region.

A still more preferred embodiment of the invention provides a method for in vitro assessing a disease condition in an individual:
i) contacting an isolated biological sample obtained from said individual with an antibody specific for a C-terminal fragment of cleaved histone H3 or an antigen-binding portion thereof as described above;
ii) providing conditions sufficient to allow said antibody specific for a C-terminal fragment of cleaved histone H3 or an antigen-binding portion thereof to bind to said isolated biological sample,
iii) determining whether the antibody specific for a C-terminal fragment of cleaved histone H3 or an antigen-binding portion thereof binds to said isolated biological sample,
   wherein said disease is associated with NET formation,
   wherein said disease is selected from the group comprising thrombotic disease, neurological disease, bacterial infection disease, viral infection disease, fungal infection disease, inflammatory disease, autoimmune disease, cancer disease, cancer metastasis disease, and

wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antibody comprises:
I) a heavy chain variable region comprising an amino acid sequence of SEQ ID NO: 1, or an amino acid sequence having a sequence identity of more than 90% with of SEQ ID NO: 1; and
II) a light chain variable region comprising an amino acid sequence of SEQ ID NO: 2, or an amino acid sequence having a sequence identity of more than 90% with of SEQ ID NO: 2,

wherein the antigen binding fragment is selected from the group comprising single chain variable fragments (scFv), variable domain (Fv) fragments, fragment antigen binding (Fab) fragments, F(ab)2 fragments, peptides, or proteolytic fragments containing an epitope binding region.

A preferred embodiment of the invention provides a method for in vitro assessing a disease condition in an individual:
i) contacting an isolated biological sample obtained from said individual with an antibody specific for a C-terminal fragment of cleaved histone H3 or an antigen-binding portion thereof as described above;
ii) providing conditions sufficient to allow said antibody specific for a C-terminal fragment of cleaved histone H3 or an antigen-binding portion thereof to bind to said isolated biological sample,
iii) determining whether the antibody specific for a C-terminal fragment of cleaved histone H3 or an antigen-binding portion thereof binds to said isolated biological sample,
   wherein said disease is associated with NET formation,
   wherein said disease is an autoimmune disease selected from the group comprising psoriasis; vasculitis; systemic lupus erythematosus (SLE); rheumatoid arthritis; ulcerative colitis; Crohn's disease; immune-mediated or Type 1 Diabetes Mellitus; immune mediated glomerulonephritis; inflammatory bowel diseases (IBD); antiphospholipid antibody syndrome; immune thrombocytopenia, and

wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8.

A preferred embodiment of the invention provides a method for in vitro assessing a disease condition in an individual:
i) contacting an isolated biological sample obtained from said individual with an antibody specific for a C-terminal fragment of cleaved histone H3 or an antigen-binding portion thereof as described above;
ii) providing conditions sufficient to allow said antibody specific for a C-terminal fragment of cleaved histone H3 or an antigen-binding portion thereof to bind to said isolated biological sample,
iii) determining whether the antibody specific for a C-terminal fragment of cleaved histone H3 or an antigen-binding portion thereof binds to said isolated biological sample,
   wherein said disease is associated with NET formation,
   wherein said disease is an autoimmune disease selected from the group comprising psoriasis; vasculitis; systemic lupus erythematosus (SLE); rheumatoid arthritis; ulcerative colitis; Crohn's disease; immune-mediated or Type 1 Diabetes Mellitus; immune mediated glomerulonephritis; inflammatory bowel diseases (IBD); antiphospholipid antibody syndrome; immune thrombocytopenia, and

wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antibody comprises:
I) a heavy chain variable region comprising an amino acid sequence of SEQ ID NO: 1, or an amino acid sequence having a sequence identity of more than 90% with of SEQ ID NO: 1; and
II) a light chain variable region comprising an amino acid sequence of SEQ ID NO: 2, or an amino acid sequence having a sequence identity of more than 90% with of SEQ ID NO: 2.

A more preferred embodiment of the invention provides a method for in vitro assessing a disease condition in an individual:
i) contacting an isolated biological sample obtained from said individual with an antibody specific for a C-terminal fragment of cleaved histone H3 or an antigen-binding portion thereof as described above;
ii) providing conditions sufficient to allow said antibody specific for a C-terminal fragment of cleaved histone H3 or an antigen-binding portion thereof to bind to said isolated biological sample,
iii) determining whether the antibody specific for a C-terminal fragment of cleaved histone H3 or an antigen-binding portion thereof binds to said isolated biological sample,
   wherein said disease is associated with NET formation,
   wherein said disease is an autoimmune disease selected from the group comprising psoriasis; vasculitis; systemic lupus erythematosus (SLE); rheumatoid arthritis; ulcerative colitis; Crohn's disease; immune-mediated or Type 1 Diabetes Mellitus; immune mediated glomerulonephritis; inflammatory bowel diseases (IBD); antiphospholipid antibody syndrome; immune thrombocytopenia, and

wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antigen binding fragment is selected from the group comprising single chain variable fragments (scFv), variable domain (Fv) fragments, fragment antigen binding (Fab) fragments, F(ab)2 fragments, peptides, or proteolytic fragments containing an epitope binding region.

A still more preferred embodiment of the invention provides a method for in vitro assessing a disease condition in an individual:
i) contacting an isolated biological sample obtained from said individual with an antibody specific for a C-terminal fragment of cleaved histone H3 or an antigen-binding portion thereof as described above;
ii) providing conditions sufficient to allow said antibody specific for a C-terminal fragment of cleaved histone H3 or an antigen-binding portion thereof to bind to said isolated biological sample,
iii) determining whether the antibody specific for a C-terminal fragment of cleaved histone H3 or an antigen-binding portion thereof binds to said isolated biological sample,
   wherein said disease is associated with NET formation,
   wherein said disease is an autoimmune disease selected from the group comprising psoriasis; vasculitis; systemic lupus erythematosus (SLE); rheumatoid arthritis; ulcerative colitis; Crohn's disease; immune-mediated or Type 1 Diabetes Mellitus; immune mediated glomerulonephritis; inflammatory bowel diseases (IBD); antiphospholipid antibody syndrome; immune thrombocytopenia, and

wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antibody comprises:
I) a heavy chain variable region comprising an amino acid sequence of SEQ ID NO: 1, or an amino acid sequence having a sequence identity of more than 90% with of SEQ ID NO: 1; and
II) a light chain variable region comprising an amino acid sequence of SEQ ID NO: 2, or an amino acid sequence having a sequence identity of more than 90% with of SEQ ID NO: 2,

wherein the antigen binding fragment is selected from the group comprising single chain variable fragments (scFv), variable domain (Fv) fragments, fragment antigen binding (Fab) fragments, F(ab)2 fragments, peptides, or proteolytic fragments containing an epitope binding region.

A preferred embodiment of the invention provides a method for in vitro assessing a disease condition in an individual:
i) contacting an isolated biological sample obtained from said individual with an antibody specific for a C-terminal fragment of cleaved histone H3 or an antigen-binding portion thereof as described above;
ii) providing conditions sufficient to allow said antibody specific for a C-terminal fragment of cleaved histone H3 or an antigen-binding portion thereof to bind to said isolated biological sample,
iii) determining whether the antibody specific for a C-terminal fragment of cleaved histone H3 or an antigen-binding portion thereof binds to said isolated biological sample,
   wherein said disease is associated with NET formation,
   wherein said disease is selected from the group comprising thrombotic disease, neurological disease, bacterial infection disease, viral infection disease, fungal infection disease, inflammatory disease, autoimmune disease, cancer disease, cancer metastasis disease, and

wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antibody specific for a C-terminal fragment of cleaved histone H3 or an antigen-binding portion thereof is linked to a detectable label.

A preferred embodiment of the invention provides a method for in vitro assessing a disease condition in an individual:
i) contacting an isolated biological sample obtained from said individual with an antibody specific for a C-terminal fragment of cleaved histone H3 or an antigen-binding portion thereof as described above;
ii) providing conditions sufficient to allow said antibody specific for a C-terminal fragment of cleaved histone H3 or an antigen-binding portion thereof to bind to said isolated biological sample,
iii) determining whether the antibody specific for a C-terminal fragment of cleaved histone H3 or an antigen-binding portion thereof binds to said isolated biological sample,
   wherein said disease is associated with NET formation,
   wherein said disease is selected from the group comprising thrombotic disease, neurological disease, bacterial infection disease, viral infection disease, fungal infection disease, inflammatory disease, autoimmune disease, cancer disease, cancer metastasis disease, and

wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antibody comprises:
I) a heavy chain variable region comprising an amino acid sequence of SEQ ID NO: 1, or an amino acid sequence having a sequence identity of more than 90% with of SEQ ID NO: 1; and
II) a light chain variable region comprising an amino acid sequence of SEQ ID NO: 2, or an amino acid sequence having a sequence identity of more than 90% with of SEQ ID NO: 2, and

wherein the antibody specific for a C-terminal fragment of cleaved histone H3 or an antigen-binding portion thereof is linked to a detectable label.

A more preferred embodiment of the invention provides a method for in vitro assessing a disease condition in an individual:
i) contacting an isolated biological sample obtained from said individual with an antibody specific for a C-terminal fragment of cleaved histone H3 or an antigen-binding portion thereof as described above;
ii) providing conditions sufficient to allow said antibody specific for a C-terminal fragment of cleaved histone H3 or an antigen-binding portion thereof to bind to said isolated biological sample,
iii) determining whether the antibody specific for a C-terminal fragment of cleaved histone H3 or an antigen-binding portion thereof binds to said isolated biological sample,
   wherein said disease is associated with NET formation,
   wherein said disease is selected from the group comprising thrombotic disease, neurological disease, bacterial infection disease, viral infection disease, fungal infection disease, inflammatory disease, autoimmune disease, cancer disease, cancer metastasis disease, and

wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antigen binding fragment is selected from the group comprising single chain variable fragments (scFv), variable domain (Fv) fragments, fragment antigen binding (Fab) fragments, F(ab)2 fragments, peptides, or proteolytic fragments containing an epitope binding region, and wherein the antibody specific for a C-terminal fragment of cleaved histone H3 or an antigen-binding portion thereof is linked to a detectable label.

A still more preferred embodiment of the invention provides a method for in vitro assessing a disease condition in an individual:
i) contacting an isolated biological sample obtained from said individual with an antibody specific for a C-terminal fragment of cleaved histone H3 or an antigen-binding portion thereof as described above;
ii) providing conditions sufficient to allow said antibody specific for a C-terminal fragment of cleaved histone H3 or an antigen-binding portion thereof to bind to said isolated biological sample,
iii) determining whether the antibody specific for a C-terminal fragment of cleaved histone H3 or an antigen-binding portion thereof binds to said isolated biological sample,
   wherein said disease is associated with NET formation,
   wherein said disease is selected from the group comprising thrombotic disease, neurological disease, bacterial infection disease, viral infection disease, fungal infection disease, inflammatory disease, autoimmune disease, cancer disease, cancer metastasis disease, and

wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antibody comprises:
I) a heavy chain variable region comprising an amino acid sequence of SEQ ID NO: 1, or an amino acid sequence having a sequence identity of more than 90% with of SEQ ID NO: 1; and
II) a light chain variable region comprising an amino acid sequence of SEQ ID NO: 2, or an amino acid sequence having a sequence identity of more than 90% with of SEQ ID NO: 2,

wherein the antigen binding fragment is selected from the group comprising single chain variable fragments (scFv), variable domain (Fv) fragments, fragment antigen binding (Fab) fragments, F(ab)2 fragments, peptides, or proteolytic fragments containing an epitope binding region, and wherein the antibody specific for a C-terminal fragment of cleaved histone H3 or an antigen-binding portion thereof is linked to a detectable label.

Another preferred embodiment of the invention provides a method as described above, wherein the antibody specific for a C-terminal fragment of cleaved histone H3 or an antigen-binding portion thereof is linked to a detectable label.

Preferably, a “detectable label” as used herein is selected from the group comprising fluorescent molecules, phosphorescent molecules, chemiluminescent molecules, bioluminescent molecules, radio-isotopes, chromophores,

Fragments and segments of histon H3 cleaved at L48R49.

The inventors have here found that the cleavage of histone H3 at site L48R49 generates two main fragments: a “N terminal fragment” of H3 comprising amino acid A1-L48 of SEQ ID NO 25, and a “C terminal fragment” of H3 comprising amino acids R49-A135 of SEQ ID NO 25.

Moreover the “N terminal fragment” of H3 cleaved at L48R49 can be further cleaved in more smaller segments during NET formation and NETosis, wherein said segments are contained in the amino acid sequence between amino acids A1-L48 of SEQ ID 25. Similarly, the “C terminal fragment” of H3 cleaved at L48R49 can be further cleaved in more smaller segments during NET formation and NETosis, wherein said segments are contained in the amino acid sequence between amino acids R49-A135 of SEQ ID NO 25.

Thus, as used herein, the term “fragment” of histone H3 refers to a fragment of histone H3 generated by the cleavage at site L48R49 of SEQ ID NO 25. Said fragment is a “N terminal fragment” of H3 or a “C terminal fragment” of H3.

As used herein the term “segment” of histone H3 refers to a segment of H3 generated by further cleavage of histone H3 or their fragments before or after cleavage at L48R49 during NET formation or NETosis.

Therefore the present invention is directed to a “N terminal fragment” of H3 comprising amino acids A1-L48 of SEQ ID NO 25 or a segment thereof, and/or to a “C terminal fragment” of H3 comprising amino acids R49-A135 of SEQ ID NO 25, or a segment thereof.

A “N-terminal fragment” of histone H3 or a segment thereof as disclosed herein is preferably between 5-48 amino acids long, preferably 10-48 amino acids long, preferably 15-48 amino acids long, preferably 20-48 amino acids long, preferably 25-48 amino acids long, preferably 30-48 amino acids long, preferably 35-48 amino acids long, preferably 5-38 amino acids long, preferably 10-38 amino acids long, preferably 15-38 amino acids long, preferably 20-38 amino acids long, preferably 25-38 amino acids long, preferably 30-38 amino acids long, preferably 5-28 amino acids long, preferably 10-28 amino acids long, preferably 15-28 amino acids long, preferably 20-28 amino acids long, preferably 25-28 amino acids long, preferably 5-18 amino acids long, preferably 10-18 amino acids long, preferably 15-18 amino acids long, preferably 2-10 amino acids long, preferably 5-10 amino acids long.

A “C-terminal fragment” of histone H3 or a segment thereof as disclosed herein is preferably between 5-87 amino acids long, preferably 10-87 amino acids long, preferably 15-87 amino acids long, preferably 20-87 amino acids long, preferably 25-87 amino acids long, preferably 30-87 amino acids long, preferably 35-87 amino acids long, preferably 45-87 amino acids long, preferably 55-87 amino acids long, preferably 65-87 amino acids long, preferably 75-87 amino acids long, preferably between 5-70 amino acids long, preferably 10-70 amino acids long, preferably 15-70 amino acids long, preferably 20-70 amino acids long, preferably 25-70 amino acids long, preferably 30-70 amino acids long, preferably 35-70 amino acids long, preferably 45-70 amino acids long, preferably 55-70 amino acids long, preferably 65-70 amino acids long, preferably between 5-60 amino acids long, preferably 10-60 amino acids long, preferably 15-87 amino acids long, preferably 20-60 amino acids long, preferably 25-60 amino acids long, preferably 30-60 amino acids long, preferably 35-60 amino acids long, preferably 45-60 amino acids long, preferably 55-60 amino acids long, preferably between 5-50 amino acids long, preferably 10-50 amino acids long, preferably 15-50 amino acids long, preferably 20-50 amino acids long, preferably 25-50 amino acids long, preferably 30-50 amino acids long, preferably 35-50 amino acids long, preferably 40-50 amino acids long, preferably between 5-40 amino acids long, preferably 10-40 amino acids long, preferably 15-40 amino acids long, preferably 20-40 amino acids long, preferably 25-40 amino acids long, preferably 30-40 amino acids long, preferably 35-40 amino acids long, preferably between 5-30 amino acids long, preferably 10-30 amino acids long, preferably 15-30 amino acids long, preferably 20-30 amino acids long, preferably between 5-20 amino acids long, preferably 10-20 amino acids long.

Therefore, a preferred embodiment of the invention provides an isolated fragment of human histone H3 cleaved at site L48R49 of SEQ ID NO 25, wherein the fragment of human histone H3 cleaved at site L48R49 consists of an amino acid sequence comprised between residues R49-A135 of SEQ ID NO 25, or a segment thereof, or consists of an amino acid sequence comprised between residues A1-L48 of SEQ ID NO 25, or a segment thereof.

As used herein the term protein or fragment thereof also include “variant” of a protein or of a fragment or segment thereof, and refers to a protein or fragment or segment that shares a certain amino acid sequence identity with the reference protein or fragment or segment upon alignment by a method known in the art. A variant of a protein or of a fragment or of a segment thereof can include a substitution, insertion, deletion, frameshift or rearrangement in another protein. In some embodiments variants share at least 70%, 80%, 85%, 90%, 95% or 99% sequence identity with the native protein or with the fragment or segment thereof.

Therefore, a more preferred embodiment of the invention provides an isolated fragment of human histone H3 cleaved at site L48R49 of SEQ ID NO 25, wherein the fragment of human histone H3 cleaved at site L48R49 consists of an amino acid sequence comprised between residues R49-A135 of SEQ ID NO 25, or a segment thereof, or consists of an amino acid sequence comprised between residues A1-L48 of SEQ ID NO 25, or a segment thereof, wherein the fragment of human histone H3 or the segment thereof share at least 70%, 80%, 85%, 90%, 95% or 99% sequence identity with the sequence of a native fragment of human histone H3 or a segment thereof.

In other words, said N-terminal fragment and said C-terminal fragment can also derive from other variants or homologues of histone H3 which comprise a sequence having at least 90% identity with SEQ ID NO 25. To notice, SEQ ID NO 25 corresponds to histone H3 sequence Q71DI3 without the first Met.

Kit for assessing a disease associated with NET formation.

In another aspect, the present invention provides a kit for assessing a disease containing an antibody specifically binding a C-terminal fragment of cleaved histone H3, or an antigen-binding portion thereof, wherein the disease is selected from the group comprising thrombotic disease, neurological disease, bacterial infection disease, viral infection disease, fungal infection disease, inflammatory disease, autoimmune disease, cancer disease, cancer metastasis disease, and wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein said disease is associated with NET formation.

In another aspect, the present invention provides a kit for assessing a disease containing an antibody specifically binding a C-terminal fragment of cleaved histone H3, or an antigen-binding portion thereof, wherein the disease is selected from the group comprising thrombotic disease, neurological disease, bacterial infection disease, viral infection disease, fungal infection disease, inflammatory disease, autoimmune disease, cancer disease, cancer metastasis disease, and wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antibody comprises:
I) a heavy chain variable region comprising an amino acid sequence of SEQ ID NO: 1, or an amino acid sequence having a sequence identity of more than 90% with of SEQ ID NO: 1; and
II) a light chain variable region comprising an amino acid sequence of SEQ ID NO: 2, or an amino acid sequence having a sequence identity of more than 90% with of SEQ ID NO: 2 and

wherein said disease is associated with NET formation.

Preferably, the antigen binding fragment is selected from the group comprising single chain variable fragments (scFv), variable domain (Fv) fragments, fragment antigen binding (Fab) fragments, F(ab)2 fragments, peptides, or proteolytic fragments containing an epitope binding region.

In another aspect, the present invention provides a kit for assessing a disease containing an antibody specifically binding a C-terminal fragment of histone H3 cleaved at L48R49 of SEQ ID NO 25, or an antigen-binding portion thereof, wherein the disease is selected from the group comprising thrombotic disease, neurological disease, bacterial infection disease, viral infection disease, fungal infection disease, inflammatory disease, autoimmune disease, cancer disease, cancer metastasis disease, and

wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8 and

wherein said disease is associated with NET formation.

In another aspect, the present invention provides a kit for assessing a disease containing an antibody specifically binding a C-terminal fragment of histone H3 cleaved at L48R49 of SEQ ID NO 25, or an antigen-binding portion thereof, wherein the disease is selected from the group comprising thrombotic disease, neurological disease, bacterial infection disease, viral infection disease, fungal infection disease, inflammatory disease, autoimmune disease, cancer disease, cancer metastasis disease, and

wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antibody comprises:
I) a heavy chain variable region comprising an amino acid sequence of SEQ ID NO: 1, or an amino acid sequence having a sequence identity of more than 90% with of SEQ ID NO: 1; and
II) a light chain variable region comprising an amino acid sequence of SEQ ID NO: 2, or an amino acid sequence having a sequence identity of more than 90% with of SEQ ID NO: 2 and

wherein said disease is associated with NET formation,

In a further aspect, the present invention provides a kit as described above, wherein the autoimmune disease is selected from the group comprising psoriasis; vasculitis; systemic lupus erythematosus (SLE); rheumatoid arthritis; ulcerative colitis; Crohn's disease; immune-mediated or Type 1 Diabetes Mellitus; immune mediated glomerulonephritis; inflammatory bowel diseases (IBD); antiphospholipid antibody syndrome; immune thrombocytopenia.

Thus, the present invention also provides a kit for assessing a disease containing an antibody specifically binding a C-terminal fragment of cleaved histone H3, or an antigen-binding portion thereof, wherein the disease is an autoimmune disease selected from the group comprising psoriasis; vasculitis; systemic lupus erythematosus (SLE); rheumatoid arthritis; ulcerative colitis; Crohn's disease; immune-mediated or Type 1 Diabetes Mellitus; immune mediated glomerulonephritis; inflammatory bowel diseases (IBD); antiphospholipid antibody syndrome; immune thrombocytopenia, and wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein said disease is associated with NET formation.

In another aspect, the present invention provides a kit for assessing a disease containing an antibody specifically binding a C-terminal fragment of cleaved histone H3, or an antigen-binding portion thereof, wherein the disease is an autoimmune disease selected from the group comprising psoriasis; vasculitis; systemic lupus erythematosus (SLE); rheumatoid arthritis; ulcerative colitis; Crohn's disease; immune-mediated or Type 1 Diabetes Mellitus; immune mediated glomerulonephritis; inflammatory bowel diseases (IBD); antiphospholipid antibody syndrome; immune thrombocytopenia, and wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antibody comprises:
I) a heavy chain variable region comprising an amino acid sequence of SEQ ID NO: 1, or an amino acid sequence having a sequence identity of more than 90% with of SEQ ID NO: 1; and
II) a light chain variable region comprising an amino acid sequence of SEQ ID NO: 2, or an amino acid sequence having a sequence identity of more than 90% with of SEQ ID NO: 2, and

wherein said disease is associated with NET formation.

Preferably, the antigen binding fragment is selected from the group comprising single chain variable fragments (scFv), variable domain (Fv) fragments, fragment antigen binding (Fab) fragments, F(ab)2 fragments, peptides, or proteolytic fragments containing an epitope binding region.

In another aspect, the present invention provides a kit for assessing a disease containing an antibody specifically binding a C-terminal fragment of histone H3 cleaved at L48R49 of SEQ ID NO 25, or an antigen-binding portion thereof, wherein the disease is an autoimmune disease selected from the group comprising psoriasis; vasculitis; systemic lupus erythematosus (SLE); rheumatoid arthritis; ulcerative colitis; Crohn's disease; immune-mediated or Type 1 Diabetes Mellitus; immune mediated glomerulonephritis; inflammatory bowel diseases (IBD); antiphospholipid antibody syndrome; immune thrombocytopenia, and wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein said disease is associated with NET formation.

In another aspect, the present invention provides a kit for assessing a disease containing an antibody specifically binding a C-terminal fragment of histone H3 cleaved at L48R49 of SEQ ID NO 25, or an antigen-binding portion thereof, wherein the disease is an autoimmune disease selected from the group comprising psoriasis; vasculitis; systemic lupus erythematosus (SLE); rheumatoid arthritis; ulcerative colitis; Crohn's disease; immune-mediated or Type 1 Diabetes Mellitus; immune mediated glomerulonephritis; inflammatory bowel diseases (IBD); antiphospholipid antibody syndrome; immune thrombocytopenia, and wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein the antibody comprises:
I) a heavy chain variable region comprising an amino acid sequence of SEQ ID NO: 1, or an amino acid sequence having a sequence identity of more than 90% with of SEQ ID NO: 1; and
II) a light chain variable region comprising an amino acid sequence of SEQ ID NO: 2, or an amino acid sequence having a sequence identity of more than 90% with of SEQ ID NO: 2, and

wherein said disease is associated with NET formation.

A further embodiment of the present invention provides a kit for assessing a disease containing a fragment of human histone H3, wherein the disease selected from the group comprising thrombotic disease, neurological disease, bacterial infection disease, viral infection disease, fungal infection disease, inflammatory disease, autoimmune disease, cancer disease, cancer metastasis disease, and wherein said fragment of human histone H3 is a fragment of human histone H3 cleaved at site L48R49 of SEQ ID NO 25, and wherein the fragment of human histone H3 cleaved at site L48R49 consists of an amino acid sequence comprised between residues R49-A135 of SEQ ID NO 25, or a segment thereof, or consists of an amino acid sequence comprised between residues A1-L48 of SEQ ID NO 25, or a segment thereof, and

wherein said disease is associated with NET formation.

A further preferred embodiment of the present invention provides a kit for assessing a disease containing a fragment of human histone H3, wherein the disease is an autoimmune disease is selected from the group comprising psoriasis; vasculitis; systemic lupus erythematosus (SLE); rheumatoid arthritis; ulcerative colitis; Crohn's disease; immune-mediated or Type 1 Diabetes Mellitus; immune mediated glomerulonephritis; inflammatory bowel diseases (IBD); antiphospholipid antibody syndrome; immune thrombocytopenia, and

wherein said fragment of human histone H3 is a fragment of human histone H3 cleaved at site L48R49 of SEQ ID NO 25, and wherein the fragment of human histone H3 cleaved at site L48R49 consists of an amino acid sequence comprised between residues R49-A135 of SEQ ID NO 25, or a segment thereof, or consists of an amino acid sequence comprised between residues A1-L48 of SEQ ID NO 25, or a segment thereof, and

wherein said disease is associated with NET formation.

Another embodiment of the present invention provides a kit for assessing a disease containing a fragment of human histone H3, wherein the disease selected from the group comprising thrombotic disease, neurological disease, bacterial infection disease, viral infection disease, fungal infection disease, inflammatory disease, autoimmune disease, cancer disease, cancer metastasis disease, and

wherein said fragment of human histone H3 is a fragment of human histone H3 cleaved at site L48R49 of SEQ ID NO 25, and wherein the fragment of human histone H3 cleaved at site L48R49 consists of an amino acid sequence comprised between residues R49-A135 of SEQ ID NO 25, or a segment thereof, or consists of an amino acid sequence comprised between residues A1-L48 of SEQ ID NO 25, or a segment thereof, and

wherein said fragment of human histone H3 or the segment thereof share at least 70%, 80%, 85%, 90%, 95% or 99% sequence identity with the sequence of native fragment of human histone H3 or a segment thereof, and

wherein said disease is associated with NET formation.

A further preferred embodiment of the present invention provides a kit for assessing a disease containing a fragment of human histone H3, wherein the disease is an autoimmune disease is selected from the group comprising psoriasis; vasculitis; systemic lupus erythematosus (SLE); rheumatoid arthritis; ulcerative colitis; Crohn's disease; immune-mediated or Type 1 Diabetes Mellitus; immune mediated glomerulonephritis; inflammatory bowel diseases (IBD); antiphospholipid antibody syndrome; immune thrombocytopenia, and

wherein said fragment of human histone H3 is a fragment of human histone H3 cleaved at site L48R49 of SEQ ID NO 25, and wherein the fragment of human histone H3 cleaved at site L48R49 consists of an amino acid sequence comprised between residues R49-A135 of SEQ ID NO 25, or a segment thereof, or

consists of an amino acid sequence comprised between residues A1-L48 of SEQ ID NO 25, or a segment thereof, and

wherein said fragment of human histone H3 or the segment thereof share at least 70%, 80%, 85%, 90%, 95% or 99% sequence identity with the sequence of native fragment of human histone H3 or a segment thereof, and

wherein said disease is associated with NET formation.

A further more preferred embodiment is directed to a kit for assessing a disease containing an antibody specifically binding a C-terminal fragment of cleaved histone H3, or an antigen-binding portion thereof, and a fragment of human histone H3, wherein the disease selected from the group comprising thrombotic disease, neurological disease, bacterial infection disease, viral infection disease, fungal infection disease, inflammatory disease, autoimmune disease, cancer disease, cancer metastasis disease, and wherein said disease is associated with NET formation, and

wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein said fragment of human histone H3 is a fragment of human histone H3 cleaved at site L48R49 of SEQ ID NO 25, and wherein the fragment of human histone H3 cleaved at site L48R49 consists of an amino acid sequence comprised between residues R49-A135 of SEQ ID NO 25, or a segment thereof, or consists of an amino acid sequence comprised between residues A1-L48 of SEQ ID NO 25, or a segment thereof, and

wherein said disease is associated with NET formation.

A further more preferred embodiment is directed to a kit for assessing a disease containing an antibody specifically binding a C-terminal fragment of cleaved histone H3, or an antigen-binding portion thereof, and a fragment of human histone H3, wherein the disease is an autoimmune disease is selected from the group comprising psoriasis; vasculitis; systemic lupus erythematosus (SLE); rheumatoid arthritis; ulcerative colitis; Crohn's disease; immune-mediated or Type 1 Diabetes Mellitus; immune mediated glomerulonephritis; inflammatory bowel diseases (IBD); antiphospholipid antibody syndrome; immune thrombocytopenia, and

wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8, and

wherein said fragment of human histone H3 is a fragment of human histone H3 cleaved at site L48R49 of SEQ ID NO 25, and wherein the fragment of human histone H3 cleaved at site L48R49 consists of an amino acid sequence comprised between residues R49-A135 of SEQ ID NO 25, or a segment thereof, or

consists of an amino acid sequence comprised between residues A1-L48 of SEQ ID NO 25, or a segment thereof; and

wherein said disease is associated with NET formation.

Use of Cleavage Site of Histone H3

A further preferred embodiment of the present invention provides a use of cleavage site of histone H3 situated at L48R49 of SEQ ID NO 25 for specific detection of neutrophil extracellular traps.

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules which generally lack antigen specificity. The terms “antibody” and “immunoglobulin” are used interchangeably in the broadest sense and include monoclonal antibodies (e.g., full length or intact monoclonal antibodies), polyclonal antibodies, human antibodies, multispecific (or heteroconjugate) antibodies (e.g. bispecific antibodies), monovalent antibodies, multivalent antibodies, antigen-binding antibody fragments such as single chain variable fragments (scFv), variable domain (Fv) fragments, fragment antigen binding (Fab) fragments, F(ab)2 fragments, peptides, or proteolytic fragments containing an epitope binding region, Fv fragments, sdAbs, VHH, antibody fusions, and synthetic antibodies (or antibody mimetics). An antibody can be chimeric, human, humanized and/or affinity matured.

As used herein, the term “antigen” is defined as any substance capable of eliciting an immune response.

As used herein, the term “immunogenicity” refers to the ability of an immunogen, antigen, or vaccine to stimulate an immune response.

As used herein, the term “epitope” is defined as the parts of an antigen molecule which contact the antigen binding site of an antibody or of a T cell receptor.

As used herein, the term “specific binding” or “specifically binding” or “binds specifically” refers to the interaction between binding pairs (e.g., an antibody and an antigen).

“Full-length antibody”, “intact antibody” or “whole antibody” are used herein interchangeably to refer to an antibody having a structure substantially similar to a native antibody structure.

“Antigen-binding portion of an antibody” refers to a portion of a full-length antibody which is capable of binding the same antigen as the full-length antibody. Examples of antibody fragments include, but are not limited to, Fv, Fab, Fab′, F(ab′)2; diabodies; linear antibodies; single-chain antibody molecules (e.g., scFv); single domain antibodies; multispecific antibodies formed from antibody fragments.

The “variable region” or “variable domain” of an antibody refers to the amino-terminal domains of heavy or light chain of the antibody. The variable domains of the heavy chain and light chain (VH and VL, respectively) of a native antibody generally have similar structures. A single VH or VL domain may be sufficient to confer antigen-binding specificity.

The term “variable” refers to the fact that certain portions of the variable domains differ extensively in sequence among antibodies and are used in the binding and specificity of each particular antibody for its particular antigen. However, the variability is concentrated in three segments called complementarity-determining regions (CDRs) or hypervariable regions (HVR) both in the light-chain and the heavy-chain variable domains. The more highly conserved portions of variable domains are called framework regions (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen-binding site of antibodies (Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity.

“Hypervariable region” or “HVR” as used herein, refers to each of the regions of an antibody variable domain which are hypervariable in sequence and/or form structurally defined loops. Generally, native antibodies comprise four chains with six HVRs; three in the heavy chain variable domains, VH (H1, H2, H3), and three in the light chain variable domains, VL (L1, L2, L3). Single domain antibodies only comprise three HVRs in the heavy chain variable domain. The HVRs generally comprise amino acid residues from the hypervariable loops and/or from the “complementarity determining regions” (CDRs). Unless otherwise indicated, HVR residues and other residues in the variable domain (e.g., FR residues) are numbered herein according to Kabat et al., 1991.

“Complementarity determining region” or “CDR” as used herein, refers to the regions within the hypervariable regions of the variable domain which have the highest sequence variability and/or are involved in antigen recognition. Generally, native antibodies comprise four chains with six CDRs; three in the heavy chain variable domains, VH (H1, H2, H3), and three in the light chain variable domains, VL (L1, L2, L3). Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35 of H1, 50-65 of H2, and 95-102 of H3 (Kabat et al., 1991).

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1 (L1)-FR2-H2(L2)-FR3-H3(L3)-FR4.

“Native antibody” refers to a naturally occurring immunoglobulin molecule. For example, native IgG antibodies are heterotetrameric glycoproteins of about 150 Daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

“Monoclonal antibody” as used herein refers to an antibody obtained from a substantially homogeneous population of antibodies, i.e., the individual antibodies comprising the population are identical and/or bind the same epitope, except for possible variant antibodies (e.g., variant antibodies contain mutations that occur naturally or arise during production of a monoclonal antibody, and generally are present in minor amounts). In contrast to polyclonal antibody preparations, which typically include different antibodies directed against different determinants (epitopes), each monoclonal antibody of a monoclonal antibody preparation is directed against a single determinant on an antigen. Thus, the term “monoclonal” indicates the character of the antibody as being obtained from a substantially homogeneous population of antibodies, and is not to be construed as requiring production of the antibody by any particular method.

“Chimeric antibody” refers to an antibody in which a portion of the heavy and/or light chain is derived from a particular source or species, while the remainder of the heavy and/or light chain is derived from a different source or species.

“Humanized antibody” refers to a chimeric antibody comprising amino acid sequences from non-human HVRs and amino acid sequences from human FRs. In certain embodiments, a humanized antibody will comprise substantially a single domain antibody, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

“Human antibody” refers to an antibody which possesses an amino acid sequence corresponding to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

“Fc region” or “Fc antibody fragment” refers to a dimer complex comprising the C-terminal polypeptide sequences of an immunoglobulin heavy chain, wherein a C-terminal polypeptide sequence is that which is obtainable by papain digestion of an intact antibody. The Fc region may comprise native or variant Fc sequences.

“Fab fragment” refers to an antibody fragment that contains a variable and constant domain of the light chain and a variable domain and the first constant domain (CH1) of the heavy chain. Papain digestion of antibodies produces two identical “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen. Fab′ fragments differ from Fab fragments by the addition of a few residues at the carboxy terminus of the heavy chain CH1 domain including one or more cysteines from the antibody hinge region. F(ab′)2 antibody fragments originally were produced as pairs of Fab′ fragments which have hinge cysteines between them. Other chemical couplings of antibody fragments also are known in the art.

“Fv fragment” refers to an antibody fragment which contains a complete antigen recognition and binding site. This region consists of a dimer of one heavy and one light chain variable domain in tight association, which can be covalent in nature, for example in scFv. It is in this configuration that the three HVRs of each variable domain interact to define an antigen binding site on the surface of the VH-VL dimer. Collectively, the six HVRs or a subset thereof confer antigen binding specificity to the antibody. However, even a single variable domain (or half of an Fv comprising only three HVRs specific for an antigen) has the ability to recognize and bind antigen, although usually at a lower affinity than the entire binding site.

“Single-chain Fv” or “scFv” refers to antibody fragments comprising the VH and VL domains of an antibody, wherein these domains are present in a single polypeptide chain. Generally, an Fv polypeptide further comprises a polypeptide linker between the VH and VL domains which enables the scFv to form the desired antigen binding structure.

An “isolated” antibody or protein or protein fragment is one which has been identified and separated and/or recovered from a component of its natural environment. Contaminant components of its natural environment are materials which would interfere with research, diagnostic or therapeutic uses for the antibody or protein or protein fragment, and may include enzymes, hormones, and other proteinaceous or nonproteinaceous solutes. Ordinarily, isolated antibody or protein or protein fragment will be prepared by at least one purification step.

The percentage of “sequence identity” is determined by comparing two optimally aligned nucleic acid or polypeptide sequences over a “comparison window” on the full length of the reference sequence. A “comparison window” as used herein, refers to the optimal alignment between the reference and variant sequence after that the two sequences are optimally aligned, wherein the variant nucleic acid or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment. Identity percentage is calculated by determining the number of positions at which the identical nucleic acid bases or amino acid residues occurs in both sequences to yield the number of matched positions, dividing the number of matched positions by the total number of positions in the reference sequence (i.e., the full length in amino acid or nucleotide) and multiplying the results by 100 to yield the percentage of sequence identity. Two nucleic acid or protein or peptide or polypeptide sequences are said to be “identical” if the sequence of nucleotides or amino acids in the two sequences is the same when optimally aligned as described above. The percentage of “sequence identity” can be determined on the comparison window defined above with the help of blastp with the “BLAST 2 Sequences” tool available at the NCBI website. (Tatusova A. et al., FEMS Microbiol Lett. 1999, 174:247-250).

Alternatively, a variant sequence may also be any amino acid sequence resulting from allowed substitutions at any number of positions of the parent sequence according to the formula below:
Ser substituted by Ser, Thr, Gly, and Asn;
Arg substituted by one of Arg, His, Gin, Lys, and Glu;
Leu substituted by one of Leu, Ile, Phe, Tyr, Met, and Val;
Pro substituted by one of Pro, Gly, Ala, and Thr;
Thr substituted by one of Thr, Pro, Ser, Ala, Gly, His, and Gin;
Ala substituted by one of Ala, Gly, Thr, and Pro;
Val substituted by one of Val, Met, Tyr, Phe, Ile, and Leu;
Gly substituted by one of Gly, Ala, Thr, Pro, and Ser;
Ile substituted by one of Ile, Met, Tyr, Phe, Val, and Leu;
Phe substituted by one of Phe, Trp, Met, Tyr, lie, Val, and Leu;
Tyr substituted by one of Tyr, Trp, Met, Phe, Ile, Val, and Leu;
His substituted by one of His, Glu, Lys, Gin, Thr, and Arg;
Gln substituted by one of Gin, Glu, Lys, Asn, His, Thr, and Arg;
Asn substituted by one of Asn, Glu, Asp, Gin, and Ser;
Lys substituted by one of Lys, Glu, Gln, His, and Arg;
Asp substituted by one of Asp, Glu, and Asn;
Glu substituted by one of Glu, Asp, Lys, Asn, Gln, His, and Arg;
Met substituted by one of Met, Phe, Ile, Val, Leu, and Tyr.

The number of amino acid deletions or substitutions in the variant sequence in comparison with the parent sequence is preferably up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acids.

“polynucleotide” or “nucleic acid molecule” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups.

“Oligonucleotide” as used herein, generally refers to short, generally single-stranded, generally synthetic polynucleotides that are generally, but not necessarily, less than about 200 nucleotides in length. The terms “oligonucleotide” and “polynucleotide” are not mutually exclusive. The description above for polynucleotides is equally and fully applicable to oligonucleotides.

The term “vector” as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced. Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors” or “recombinant vectors”). In general, expression vectors of utility in recombinant DNA techniques are often in the form of plasmids. In the present specification, “plasmid” and “vector” may be used interchangeably as the plasmid is the most commonly used form of vector.

The nucleic acid molecules may comprise a native sequence (i.e., an endogenous sequence that encodes an antibody or a portion thereof) or may comprise a variant of such a sequence.

Nucleic acid variants contain one or more substitutions, additions, deletions and/or insertions such that the immunoreactivity of the encoded antibody is not substantially different, relative to a native antibody of reference. In some embodiments, nucleic acid variants exhibit at least about 70% identity, in some embodiments, at least about 80% identity, in some embodiments, at least about 85% identity, in some embodiments, at least about 90% identity, and in some embodiments, at least about 95% identity to the parent nucleic acid sequence, wherein the percentage of sequence identity is determined as described above. These amounts are not meant to be limiting, and increments between the recited percentages are specifically envisioned as part of the disclosure.

The nucleic acid molecules of this disclosure can be obtained using chemical synthesis, recombinant methods, or polymerase chain reaction (PCR). Methods of chemical polynucleotide synthesis are well known in the art and need not be described in detail herein. One of skill in the art can use the sequences provided herein and a commercial DNA synthesizer to produce a desired DNA sequence. For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art.

Suitable cloning vectors and expression vectors can include a variety of components, such as promoter, enhancer, and other transcriptional regulatory sequences. The vector may also be constructed to allow for subsequent cloning of an antibody variable domain into different vectors. Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as Merck, BioRad, Strategene, and Invitrogen.

Further, the present invention also envisages expression vectors comprising nucleic acid sequences encoding any of the single domain antibodies here disclosed or variants thereof, as well as host cells comprising such expression vectors. It is implied that an expression vector must be replicable in the host cells either as episomes or as an integral part of the chromosomal DNA. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the nucleic acid molecules of interest and/or the nucleic acid molecules themselves, can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of the particular procedure will often depend on features of the host cell.

As used herein, the term “in vitro” or “ex vivo” refers to an artificial environment and to processes or reactions that occur within an artificial environment, for example, but not limited to, test tubes and cell cultures. The term “in vivo” refers to a natural environment (e.g., an animal or a cell) and to processes or reactions that occur within a natural environment.

An “autoimmune disease”, as pertains to the present invention, is a disease or disorder arising from and directed against an individual's own tissues or a co-segregate or manifestation thereof or resulting condition therefrom. This class of disorders is highly varied, both between and within different kinds of autoimmune diseases, which complicates diagnosis and effective treatment. The causes of autoimmune diseases are also poorly understood, which results in courses of treatment that focus primarily on the symptoms.

A disease associated with NET formation is preferably selected from the group comprising thrombotic disease, neurological disease, bacterial infection disease, viral infection disease, fungal infection disease, inflammatory disease, autoimmune disease, cancer disease, cancer metastasis disease.

An “autoimmune disease” associated with NET formation is preferably selected from the group comprising psoriasis; vasculitis; systemic lupus erythematosus (SLE); rheumatoid arthritis; ulcerative colitis; Crohn's disease; immune-mediated or Type 1 Diabetes Mellitus; immune mediated glomerulonephritis; inflammatory bowel diseases (IBD); antiphospholipid antibody syndrome; immune thrombocytopenia.

“Systemic Lupus Erythematosus” (SLE) is a debilitating autoimmune disease that can damage multiple organs, induce chronic renal failure, and lead to severe morbidity and mortality. A characteristic feature of SLE is the presence of anti-nuclear autoantibodies that form immune complexes with cellular debris and cause end-organ damage. Current treatment regimens are limited to non-specific immune suppression and management of inflammatory symptoms.

The term “lupus” as used herein is an autoimmune disease or disorder that in general involves antibodies that attack connective tissue. The principal form of lupus is a systemic one, systemic lupus erythematosus (SLE); including cutaneous SLE and subacutecutaneous SLE, as well as other types of lupus including nephritis, extrarenal, cerebritis, pediatric, non-renal, discoid, and alopecia. In certain embodiments, the term “systemic lupus erythematosus” refers to a chronic autoimmune disease that can result in skin lesions, joint pain and swelling, kidney disease (lupus nephritis), fluid around the heart and/or lungs, inflammation of the heart, and various other systemic conditions. In certain embodiments, the term “lupus nephritis” refers to inflammation of the kidneys that occurs in patients with SLE. Lupus nephritis may include, for example, glomerulonephritis and/or interstitial nephritis, and can lead to hypertension, proteinuria, and kidney failure. Lupus nephritis classes include class I (minimal mesangial lupus nephritis), class II (mesangial proliferative lupus nephritis), class 1II (focal lupus nephritis), class IV (diffuse segmental (IV-S) or diffuse global (IV-G) lupus nephritis), class V (membranous lupus nephritis), and class VI (advanced sclerosing lupus nephritis).

“Cancer” and “cancerous” refer to, or describe a physiological condition in mammals that is typically characterized by a cell proliferative disorder. Cancer generally can include, but is not limited to, carcinoma, lymphoma (e.g., Hodgkin's and non-Hodgkin's lymphoma), blastoma, sarcoma, and leukemia. More specific examples of cancer can include, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, leukemia and other lymphoproliferative disorders, and various types of head and neck cancer.

As used herein, the term “individual”, “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. Preferably, the subject is a human patient that is at for, or suffering from, an autoimmune disease.

### DESCRIPTION OF THE FIGURES

FIG. 1 shows that PMA induced Histone H3 cleavage occurs in the N-terminal domain and is prevented by serine protease inhibition.
(A) Neutrophils were preincubated with the serine protease inhibitor AEBSF for 30 min in microcentrifuge tubes and then stimulated with PMA as indicated in the figure. Lysates were resolved by SDS-PAGE and immunoblotted with N- and C-terminal antibodies to H3. GAPDH was used as a loading control. The data show that serine proteases cleave the N-terminal end of H3 at specific amino-acids.
(B) Immunofluorescent confocal microscopy of NET formation. Neutrophils were seeded on coverslips and preincubated with AEBSF before stimulation with PMA (50 nM) as indicated in the figure. At 150 min the cells were fixed and stained for neutrophil elastase (NE), chromatin (using a H2A-H2B-DNA antibody PL2.3) and DNA using DAPI (4′,6-diamidino-2-phenylindole). NS: non-stimulated. Images were taken at 63× and the scale bar is 20 μm.

FIG. 2 shows the identification of histone H3 cleavage sites in NET formation.
(A) Representative RP-HPLC chromatogram of acid extracted histones from NETs and corresponding 1 D-SDS-PAGE and immunoblots to identify H3 and H4 containing fractions. Histone enriched supernatants were prepared from neutrophils stimulated with PMA for 90 min.
(B) Representative Coomassie stained blot of pooled H3 fractions separated by 2D-electrophoresis. Inset is a zoom of all spots (1-5) identified as histone H3 by mass spectrometry.
(C) Schematic representation of the cleavage site of the truncated H3 product in both the linear sequence of H3 and in the nucleosomal context. H3 is represented in blue and the pink tail region and partial alpha helix represent the part of H3 that is removed. The nucleosome structure is adapted from PDB 2F8N. The structure is assembled by X-ray crystallography and it is made of histones from multiple species. The H3 variant is H3.2 of Xenopus but it has 100% identity with Human H3.2. Protein identifiers P84233 and Q71 DI3 for Xenopus and Human respectively.

FIG. 3 shows the screening and detection of cleaved H3 and NETs by 3D9.
(A) Immunofluorescent microscopy of neutrophils stimulated with PMA and fixed at the indicated times. Samples were stained with Hoechst (DNA—blue) and 3D9 (with Alexafluor-568 conjugated secondary antibody—red). NS: non-stimulated. Images were taken on an upright fluorescent microscope at 20×. Scale bars-50 μm (full field) and 10 μm (inset).
(B) Immunoblots of lysates prepared from neutrophils stimulated with PMA (50 nM) for the times indicated in the figure. H3 C-terminal antibody was used as a control to detect all H3 forms while a single band (cleaved H3) was detected by the newly generated monoclonal antibody, 3D9.
(C) Direct ELISA for cleaved H3 in NETs, chromatin (A549 lung epithelial cells), recombinant histone H3 and DNA. Samples were serially diluted and immobilized on a high affinity ELISA plate according to DNA content (for NETs, chromatin and DNA) or protein content (for recombinant histone H3) as determined by PicoGreen and bicinchoninic acid assays respectively. Starting concentration was 1 μg/ml DNA or protein. Cleaved H3 was detected using 3D9 (2 μg/ml) and HRP conjugated anti-mouse secondary antibody and reactions were developed using TMB (3,3′,5,5′-tetramethylbenzidine) as a substrate. Data is presented for dilution 200 ng/ml. REIRR indicates unconjugated branched immunising peptide control formed by SEQ ID NO 11 and SEQ ID NO 26. This peptide was coated at 20 ng/ml. Data represents mean±SD of 3 experiments using independent donors.
(D) Shows the ELISA and subsequent inhouse immunoblot and immunofluorescent microscopy screening strategy. (E) Immunofluorescent microscopy of neutrophils stimulated with PMA for 3 h and stained with 3D9 (1 μg/ml) in the presence of competition and control peptides. Prior to staining, 3D9 was preincubated overnight at 4° C. with immunising branched peptide (SEQ ID NO 11 and 12) and negative control branched peptide (SEQ ID NO 16 and 17) at the indicated ratio of molar concentration. (F, G) Neutrophils were seeded in 10 ml petri dishes and stimulated for 3 h with PMA. Before total cell lysis and immunoprecipitation, NETs were digested with benzonase to liberate histones and octamers for antibody binding. Line 1: ISO, Line 2: 3D9, Line 3: Beads only; INP: input; M: marker. (F) Coomassie stained gel of proteins immunoprecipitated by 3D9 or isotype control and the corresponding silver stained gel. The predicted cleaved H3 and intact H3 bands are indicated. HC-Ab: heavy chain of antibody, LC-Ab: light chain of antibody. (G) Immunoblot for total H3 (C-terminal antibody) and cleaved H3 of the proteins immunoprecipitated by 3D9 or isotype control antibodies and Coomassie stained gel of samples run in parallel. Gels are representative of 3 independent experiments.

FIG. 4 shows the comparison of NET quantification with an anti-chromatin antibody PL2.3 versus 3D9.
(A) Confocal immunofluorescent microscopy of neutrophils stimulated with PMA (180 min) and stained with Hoechst and anti-chromatin antibody (PL2.3) or 3D9. Insets represent selected cells examined at higher magnification (63×) and presented as split channels in grayscale or merged as per the total field of view (20× magnification). White scale bar—50 μm, cyan scale bar—10 μm. Images are representative of 3 experiments.
(B) Comparison of the fluorescent distribution of PL2.3 versus 3D9 staining of PMA stimulated cells over time (6 h). Staining intensities were normalized over all images of the respective time course. NS:360: non-stimulated at 360 min. Analysis is performed on one data set that is representative of 3-4 independent time course experiments.
(C) Comparison of NET quantification using manual or automatic thresholding and segmentation procedures for chromatin antibody (PL2.3) and cleaved H3 antibody (or 3D9). Manual thresholding excludes cells/NETs with a weak signal whereas automatic thresholding includes all objects irrespective of signal. Images for analysis were taken using a fluorescence microscope. Graph represents the mean±standard deviation, where n=3-4.

FIG. 5 shows the detection by 3D9 of NETs from diverse stimuli.
Immunofluorescence microscopy of neutrophils left unstimulated (NS), stimulated with PMA (50 nM, 2.5 h), nigericin (15 μM, 2.5 h), TNF primed and then stimulated with heme (20 μM, 6 h), and neutrophils co-cultured with Candida albicans hyphae (MOI 5) for 4 h. Samples were stained with Hoechst, anti-neutrophil elastase (NE) and 3D9. Scale bar—50 μm. Images were taken on a confocal microscope at 20× and are representative of 3 experiments with independent donors. Scale bar—20 μm.

FIG. 6 shows the detection of NETs in mixed cell fractions.
Immunofluorescence microscopy of non-purified peripheral blood mononuclear cell (PBMC) fractions treated with the NET stimuli, PMA (50 nM, 2.5 h) or nigericin (15 μM, 2.5 h), and then stained with Hoechst, anti-neutrophil elastase (NE) and 3D9 or PL2.3. Images were taken on an upright fluorescent microscope at 20× magnification and the selected images are representative of 3 independent experiments. Scale bar—50 μm.

FIG. 7 shows the comparison of 3D9 detection in response to apoptotic, necroptotic and necrotic cell death stimuli.
A) Confocal immunofluorescent microscopy of neutrophils stimulated with different cell death stimuli and subsequently stained with Hoechst, anti-neutrophil elastase (NE) and 3D9. NETs were induced with PMA (100 nM, 3 h). Apoptosis was induced in resting neutrophils by incubation for 24 h without stimulation overnight. Neutrophils were stimulated with caspase inhibitor Z-VAD-FMK (50 μM) plus SMAC mimetic (100 nM) plus TNF (50 ng/ml) for 6 h to induce necroptosis. Necrosis was induced with the pore forming toxin α-haemolysin (25 μg/ml). Images were taken at 20× and are representative of 3 experiments. Scalebar 20 μm. ns: non stimulated.
B) A comparison was made with parallel samples stained with the chromatin antibody PL23. ns: non stimulated.

FIG. 8 shows the detection clipped histone 3 and NETs in human tissues.
Paraffin embedded sections were stained with Hoechst, anti-NE and 3D9 or H3 cit antibodies and examined by confocal microscopy. Scale bar—10 μm (A) inflamed human tonsil (B & C) Inflammed human kidney.

FIG. 9 shows the comparison of clipped H3, H3cit and H2B staining in the gall bladder.
Paraffin embedded sections were stained with Hoechst, anti-NE and histone antibodies 3D9, H3cit and H2B and examined by confocal microscopy. Scale bar—20 μm.

FIG. 10 shows the comparison of clipped H3, H3 cit and H2B staining in the appendix. Paraffin embedded sections were stained with Hoechst, anti-NE and 3D9 or H3cit antibodies and examined by confocal microscopy. Scale bar—20 μm.

The following examples are included to demonstrate preferred embodiments of the invention. It should be appreciated by those of skill in the art that the techniques disclosed in the following examples represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

### EXAMPLES

Materials and Methods

Reagents

All reagents were purchased from common vendors of laboratory reagents e.g. Sigma Aldrich or VWR Deutschland unless otherwise stated.

Blood Collection and Ethical Approval

Venous blood was collected from healthy donors who had provided informed consent according to the Declaration of Helsinki. Ethical approval was provided by the ethics committee of Charité-Universitätsmedizin Berlin and blood was donated anonymously at Charité Hospital Berlin.

Purification and Culture of Human Peripheral Blood Neutrophils

Neutrophils were isolated as described by Amulic et al (2017). Briefly, venous whole blood was collected in EDTA and separated by layering over equal volume Histopaque 1119 and centrifugation at 800 g (20 min). The pinkish neutrophil rich fraction was collected and washed once by the addition of 3 volumes of wash buffer (PBS, without Mg²⁺ or Ca²⁺[Gibco] supplemented with 0.5% [w/v] human serum albumin [HSA, Grifols]) and centrifugation at 300 g (10 min). The neutrophil fraction was further purified by density gradient centrifugation—Percoll (Pharmacia) gradient from 85%-65% (v/v). Purified cells were collected from the 80-70% fractions and washed once before being resuspended in wash buffer. Cells were counted using a CASY cell counter.

For all experiments, unless indicated, neutrophils were cultured RPMI (GIBCO 32404014) supplemented with 10 mM HEPES and 0.1% (w/v) HSA, which had been preequilibrated in CO₂ conditions for 1 h. For some stimuli, the HSA content was reduced to 0.05% or 0% HSA as indicated in the figure legends. Cells were routinely cultured at 37° C., 5% CO₂ unless indicated. For all experiments, stimuli were added to cell reactions as 10× working stock solutions freshly diluted in RPMI. For inhibition experiments, a 10× inhibitor stock and appropriate vehicle controls, were added to the cells and preincubated for the times stated in the figure legends.

Neutrophil and NET Lysate Preparation

To analyse proteins, lysates were prepared from stimulated or resting neutrophils. Cells were seeded in culture medium in 1.5 ml microcentrifuge tubes at 1×10⁷ cells/ml with 5×10⁶ cells per time point. After addition of the inhibitor or agonist, cells were gently mixed and incubated at 37° C. with gentle rotation. At the specified time points, protease inhibitors—1 mM AEBSF, 20 μM Cathepsin G inhibitor I (Calbiochem), 20 μM neutrophil elastase inhibitor GW311616A (Biomol), 2× Halt protease inhibitor cocktail (PIC, Thermofisher Scientific), 10 mM EDTA, 2 mM EGTA—were added directly to the cell suspension. Cells were gently mixed and centrifuged at 1000 g (30 s) to collect all residual liquid. Boiling 5× sample loading buffer (50 mM Tris-HCl pH 6.8, 2% [w/v] SDS, 10% glycerol, 0.1% [w/v] bromophenol blue, 100 mM DTT) was added to samples which were then briefly vortexed and boiled (98° C.) for 10 min and flash frozen in liquid nitrogen for storage at −80° C.

1D SDS-PAGE and Immunoblot Blot

For routine protein analysis, samples were analysed by 1D SDS-PAGE and immunoblotted. Samples were thawed on ice, boiled at 98° C. (10 min) and sonicated to reduce viscosity (Braun sonicator, 10 s, cycle 7, power 70%). Proteins were applied to NuPAGE 12% gels (Invitrogen, Thermofisher) and run at 150 V in MES buffer (Thermofisher Scientific). Proteins were transferred by western blot onto PVDF (0.2 μm pore size, Amersham GE Healthcare) using the BioRad wet transfer system (buffer: 25 mM Tris, 192 mM glycine, 20% methanol, protocol: 30 min at 100 mA, 120 min at 400 mA). Blotting efficacy was assessed by Ponceau S staining. Blots were blocked with TBST (TBS pH 7.5, 0.1% [v/v] Tween-20) with 5% [w/v] skimmed milk, for 1 h at RT. Blots were then incubated with the following primary antibodies overnight at 4° C. or for 2 h at RT: rabbit anti-histone H3 C-terminal pAb, 1:15000 (Active motif #61277); rat anti-histone H3 N-terminal mAb, 1:1000 (Active Motif #61647, aa 1-19); Histone H4 C-terminal, 1:5000 (Abcam 10158— aa 50 to C terminal); rabbit anti-histone H4 N-terminal mAb, 1:30,000 (Upstate, Millipore #05-858, aa17-28); rabbit GAPDH mAb, 1:5000 (Cell Signalling Technology, #2118); mouse 3D9 1 ug/ml (produced in this study)—all diluted in TBST with 3% (w/v) skimmed milk. After washing with TBST (3×5 min), blots were blocked for 15 min as before and then probed with secondary HRP conjugated antibodies (Jackson Immuno Research—diluted 1:20000 in 5% skimmed milk TBST) for 1 h at RT. Blots were washed in TBST (3×5 min) and developed using SuperSignal™ West Dura Extended Duration Substrate (ThermoFisher Scientific) and an ImageQuant Gel imager (GE Healthcare).

Immunofluorescent Staining of In Vitro Samples

For immunofluorescent imaging of purified cells, neutrophils/PBMCs were seeded in 24 well dishes containing glass coverslips, with 1×10⁵ cells per well and incubated at 37° C. for 1 h allowing to adhere to the coverslip. At this stage, inhibitors and priming factors were included as indicated. Reactions were stopped by the addition of paraformaldehyde (2% [w/v]) for 20 min at RT or 4° C. overnight. After fixation, cells were washed and stained as previously described (Brinkmann et al 2012). Briefly, all steps were performed by floating inverted coverslips on drops of buffer on laboratory parafilm. Cells were permeabilised with PBS, pH 7.5, 0.5% (v/v) Triton X-100 for 3 min. For screening and quantification experiments, this permeabilization step was extended to 10 min. Samples were washed (3×5 min) with PBS, 0.05% (v/v) Tween 20 and incubated with blocking buffer—PBS pH 7.5, 0.05% (v/v) Tween 20, 3% (v/v) normal goat serum, 3% (w/v) freshwater fish gelatin, 1% (w/v) BSA—for 20 min at RT and then probed with primary antibodies diluted in blocking buffer and incubated overnight at 4° C. Primary antibodies: anti-chromatin (H2A-H2B-DNA complex) mouse mAb, 1 μg/ml (Losman 1992); neutrophil elastase, rabbit pAb 1:500 (Calbiochem); mouse serum for screening, 1:100; hybridoma supernatants, neat; 3D9 mouse mAb, 1 μg/ml. Samples were then washed as before. Alexa labelled secondary antibodies (Invitrogen) were diluted 1/500 in blocking buffer and incubated for 2 h at RT. DNA was stained with Hoescht 33342 (Invitrogen, Molecular Probes) 1 ug/ml, incubated with the secondary antibody step. Samples were washed in PBS followed by water and mounted in Mowiol mounting medium.

Histone Extraction from Neutrophils

Histone enriched fractions were prepared from resting neutrophils and NETs according to a method modified from Shechter et al. (Shechter, D., Dormann, H. L., Allis, C. D., and Hake, S. B. (2007). Extraction, purification and analysis of histones. Nat Protoc 2, 1445-1457). Neutrophils (4-8×10⁷) were resuspended in 13 ml of RPMI (without HSA) in a 15 ml polypropylene tube and incubated on a roller at 37° C. with PMA 50 nM for 90 min. After stimulation, 1 mM AEBSF was added to inhibit further degradation by NSPs and cells were cooled on ice for 10 min. All subsequent steps were performed on ice or 4° C. where possible. Cells and NETs were pelleted by centrifugation at 1000 g, 10 min. Samples were resuspended in ice-cold hypotonic lysis buffer (10 mM Tris-HCl pH 8.0, 1 mM KCL, 1.5 mM MgCl₂, 1 mM DTT supplemented with protease inhibitors just before use—1 mM AEBSF, 20 μNA NEi, 20 μNA CGi, 2×PIC, 10 mM EDTA) using 1 ml of buffer/5×10⁶ cells. Cells were then incubated at 4° C. on a rotator for 30 min before being passed through a syringe to aid lysis and shearing of intact cells. Nuclei and NETs were collected by centrifugation at 10,000 g, 10 min, discarding the supernatant. To disrupt nuclei, samples were resuspended in dH₂O (1 ml/1×10⁷ cells) supplemented with protease inhibitors, as before, and incubated on ice for 5 min with intermittent vortexing. NP40 (0.2% [v/v]) was added to help lysis and disruption of NETs and samples were sonicated briefly (10 s, mode 7, power 70%). To extract histones, H2504 (0.4 N) was added to samples, and vortexed briefly. Samples were then incubated, rotating, for 2-3 h. Histone enriched fractions were collected by aliquoting samples into multiple 1.5 ml microcentrifuge tubes and centrifuging at 16,000 g for 10 min, followed by collection of the supernatants. To minimise further processing of histones, proteins were immediately precipitated overnight by dropwise addition of trichloroacetic acid to a final concentration of 33% followed by mixing. The next day precipitated proteins were pelleted by centrifugation at 16,000 g, 10 min. The supernatants were discarded and waxy pellets were washed once with equal volume ice-cold acetone with 0.2% (v/v) HCl and 5 times with ice-cold acetone alone. Pellets were allowed to air dry for 5 min before being resuspended with 1 ml (per 5×10⁶ cells) of dH₂O plus 1 mM AEBSF. For difficult to resuspend pellets the mixture was vigorously shaken at 4° C. overnight before samples were centrifuged, as before, to remove undissolved protein. Pooled supernatants for each sample were lyophilised and stored at −80° C. until histone fractionation.

Purification of Histone H3

Histones were fractionated by RP-HPLC according to a previously described method. After lyophilisation samples were resuspended in 300 μl Buffer A (5% acetonitrile, 0.1% trifluoroacetic acid) and centrifuged at 14,000 g to remove particulate matter. 150 μl of clarified sample was mixed with 40 μl Buffer A before being applied to a C18 column (#218TP53, Grave Vydac) and subjected to RP-HPLC (Waters 626 LC System, MA, US) as described by Schechter et al. (Nat. Protoc. 2007, 2, 1445-1457). The flow rate was set to 1 ml min⁻¹ and fractions were collected at 30 s intervals from minute 30 to 55. All fractions were lyophilised and stored at −80° C. until analysis. To determine which fractions contained H3 and cleaved species, each fraction was dissolved in 50 μl dH₂O and 5 μl was subjected to SDS-PAGE and either stained with Coomassie blue stain or transferred to PVDF and immunoblotted for H3 and H4 as described already.

2D Electrophoresis (2-DE) of Purified Histones

To determine the cleavage sites, H3 containing fractions were pooled and subjected to a modified 2-DE procedure as previously described. Briefly, pooled fractions were denatured in 9 M urea, 70 mM DTT, 2% Servalyte 2-4 and 2% Chaps. Samples (30 μl) were applied to 1.5 mm thick isoelectric focusing (IEF) gels and a shortened IEF protocol was used: 20 min 100 V, 20 min 200 V, 20 min 400 V, 15 min 600 V, 5 min 800 V, and 3 min 1000 V, (a total of 83 min and 500 V/h) in 8 cm long IEF tube gels. Separation in the second dimension was performed in 6.5 cm×8.5 cm×1.5 mm SDS-PAGE gels. Duplicate gels were prepared; one stained with Coomassie Brilliant Blue R250 for excision of spots for mass spectrometry identification; and the second transferred to PVDF as follows. Proteins were blotted onto PVDF blotting membranes (0.2 μm) with a semidry blotting procedure in a blotting buffer of 100 mM borate. Spots were stained by Coomassie Brilliant Blue R250 and analysed by N-terminal Edman degradation sequencing (Proteome Factory, Berlin, Germany).

Antibody Generation

Immunising and screening peptides are the following: branched peptide formed by REIRR (SEQ ID NO 11) and REIRRKC-NH₂ (SEQ ID NO 12) conjugated to KLH for immunization, branched peptide formed by AARKS (SEQ ID NO 13) and AARKSKC-NH₂ (SEQ ID NO 14) for negative screening, competing branched peptide formed by SEQ ID NO 11 and REIRRKC-NH₂ (SEQ ID NO 15) for validation, and branched peptide formed by TGGVK (SEQ ID NO 16) and TGGVKK-NH₂ (SEQ ID NO 17) as negative control for validation, SEQ ID NO 11 and REIRRKC-NH₂ (SEQ ID NO 26), not conjugated to KLH, for positive screening and Elisa. In the branched peptides, the first peptide (SEQ ID NO 11, SEQ ID NO 13, or SEQ ID NO 16) is bound via amide linkage between the carboxyl end of the amino acid in position 5 and the side chain amine of lysine in position 6 of the second peptide (SEQ ID NO 12, SEQ ID NO 14, SEQ ID NO 15, SEQ ID NO 17, or SEQ ID NO 26). These peptides were synthesized by Eurogentec (Belgium). A portion was further conjugated to Key Lymphocyte Haemagluttinin (KLH) for immunization. Immunisation of mice, preliminary ELISA screening and production of hybridomas were performed by Genscript as follows. Six mice (3× Balb/c and 3× C57/BL6) were immunized with branched peptides. Mice were bled and effective immunization was assessed using a direct ELISA. The ELISA and subsequent inhouse immunoblot and immunofluorescent microscopy screening strategy are outline in FIG. 3D. Following selection of effectively immunized animals, a further boost injection of the immunogen was given before isolation of spleen cells for hybridoma production. The resulting hybridoma supernatants were screened similarly. Large-scale culture of supernatants and purification of antibodies was performed by Genscript.

ELISA

To assess 3D9 specificity for NETs, 3D9 was used in an indirect ELISA to detect cleaved H3 in purified NETs, chromatin, recombinant H3 and DNA. NETs were prepared by seeding 3×10⁶ neutrophils in a 6 well dish and incubating for 3-6 h with 100 nM PMA. NETs were gently washed 3 times with equal volume PBS, before being collected in 300 μl PBS. Clumped NETs were disrupted by sonicating briefly (3 s, mode 7, power 70%—Braun Sonicator) and then snap frozen and stored at −80° C. Chromatin was prepared from lung epithelial cells (A549) as previously described by Shechter et al. (Nat. Protoc. 2007, 2, 1445-1457). The final nuclear pellet was resuspended in dH₂O and sonicated as before and stored at −80° C. The DNA content of NETs and chromatin was assessed by PicoGreen assay according to the manufacturer's instructions (Thermofisher Scientific). Beginning at 1 μg/ml (of DNA content), serial dilutions of NETs, chromatin and calf thymus DNA (Invitrogen) were prepared in lo-DNA bind Eppendorf microcentrifuge tubes. A similar dilution series of recombinant histone H3 (New England Biolabs) was prepared starting at 1 μg/ml protein. All dilutions were performed in PBS. One hundred microliters of each sample, in duplicate, at dilutions 1 ug/ml to 1 ng/ml, was aliquoted in a Nunc Maxisorb 96 well dish and immobilised overnight at 4° C., 250 rpm. The immunising peptide, REIRR (10 ng/ml) was used as a positive control. The following day all wells were washed 6 times with wash buffer (PBS, 0.05% Tween 20) and then blocked with 200 μl of blocking solution (1% BSA in wash buffer) for 2 h (RT). Wells were washed once with wash buffer and incubated with 100 μl of 3D9 (2 μg/ml, in blocking solution) at RT (2 h) with gentle shaking (250 rpm). Wells were washed 6 times as before and then incubated with 100 μl of secondary HRP conjugated anti-mouse (Jackson laboratories) at 1:10,0000 dilution in blocking solution and incubated as before. Finally, wells were washed 6 times as before and HRP activity was detected using TMB (3,3′, 5,5′ tetramethylbenzidine) reagent (BD OptEIA™) according to the manufacturer's instructions (incubating for 15-30 min). The assay was stopped by the addition of 100 μl H₂SO₄ (0.16 M) and absorbance (450 nm) was measured on a 96 well plate reader (VERSAmax, Molecular Devices, CA, US).

Quantification of staining characteristics by Image J and R In order to assess staining characteristics of antibodies during NET formation the inventors developed a bundle of Image J and R scripts. These scripts allow for an automated workflow starting from 2-channel microscopic images of an experimental series (DNA stain, antibody stain), to a graphical representation and classification of individual cells and eventually to mapping these classifications back to the original images as a graphical overlay. In the first step nuclei are segmented based on intensity thresholding (either programmatic or manual), including options for lower and upper size selection limits. The same threshold is applied to the entire experimental series and the upper size limit is used to exclude fused structures that cannot be assigned individual cells. A quality score is assigned to every image based on the fraction of the total DNA stained area that can be assigned to individual cells (or NETs). This score along with all other parameters of the analysis is exported as report file and can be used to automatically exclude images from the analysis. In addition, this part of the script generates a result file that includes the area, circumference (as x,y coordinates) and cumulative intensities for each channel for every detected nucleus along with information such as time point or stimulus that can be assigned programmatically. In order to analyse these data sets we implemented a series of functions in R. These functions include import of Image J result files, classification of cells based on nuclear area and staining intensity, various plot and data export functions, as well mapping functions that allow to display the classification of nuclei as color coded circumferences overlaid to the original image.

Quantification of NETs by Image J

NETs were quantified by the semi-automatic method described by Brinkmann et al. (Brinkmann, et al. (2012). Automatic quantification of in vitro NET formation. Frontiers in immunology 3, 413) and via a second modified method that allowed automatic quantification. All microscopy image datasets were processed by both methods to allow comparison. Hoechst was used to stain total DNA and NETs were additionally stained with anti-chromatin (PL2.3) or 3D9 antibodies (1 μg/ml) and Alexa-568 coupled secondary antibody according to the previous section. Images were acquired with a Leica DMR upright fluorescence microscope equipped with a Jenoptic B/W digital microscope camera and 10× or 20× objective lens. For each experiment, the same exposure settings were used for all samples and a minimum of 3 random fields of view (FOV) were collected. Images were analysed using ImageJ/FIJI software. As described by Brinkmann et al. 2012, each channel was imported as an image sequence and converted into a stack. To count total cells/NETs per fields of view (FOV), the Hoechst channel stack was imported and segmented using the automatic thresholding function (Bernsen method) with radius 15 and parameter 1 set to 35 to produce a black and white thresholded image. Particle analysis was then performed to count all objects the size of a cell nucleus or bigger (10× objective: particle size 25-infinity; 20× objective: particle size 100-infinity) and to exclude background staining artefacts. Total NETs were then counted using the anti-chromatin (PL2.3) or 3D9 channels accordingly. For the above method, anti-chromatin stains were segmented using manually adjusted thresholding so that the less intense staining of resting cell nuclei was excluded. Particle analysis was then performed to count all objects larger than a resting cell nucleus (10× objective: particle size 75-infinity; 20× objective: particle size 250-infinity). This was also performed for 3D9. The second automatic NET analysis was performed as the manual semi-automatic one with the exception that the workflow was modified so that the automatic Bernsen thresholding and segmentation were used for both Hoechst and NET channels, total cells and NETs, respectively. For each method, percentage NETs per FOV were calculated as (Total NETs per FOV/Total cells per FOV)×100. Results per FOV were then averaged according to sample.

Immunofluorescent Staining of Histological Samples from Tissue Sections

Paraffin sections (2 μm thick) were deparaffinized in two changes of 100% xylene for 5 min each and then rehydrated in two changes of 100% ethanol for 5 min each and followed by 90% and 70% ethanol for 5 min each. Sections were washed with 3 changes of deionized water and incubation in TBS (Tris buffered saline). For antigen retrieval, Target Retrieval Solution (TRS pH9) (Dako S2367) was used to incubate the slides in a steam cooker (Braun) for 20 min. After cooling down to room temperature in antigen retrieval buffer, slides were rinsed 3× in deionized water and incubated in TBS until further processing. Slides were blocked with blocking buffer (1% BSA, 5% normal donkey serum, 5% cold water fish gelatin and 0.05% Tween20 in TBS, pH7,4) for 30 min. Blocking buffer was removed, and sections were incubated with primary antibodies at appropriate dilution in blocking buffer (containing 0.05% Triton-X100) overnight at RT. Sections were rinsed in TBS and then incubated with secondary antibody at an appropriate concentration (1:100) for 45 min in the dark at RT and then rinsed three times in TBS for 5 min followed by rinsing with deionized water. Slides were incubated with DNA stain Hoechst 33342 (1:5000) for 5 min, rinsed with water before mounting with Mowiol. Primary antibodies for detection of NETs were as follows: mouse anti-cleaved histone 3 clone 3D9 (2 μg/ml), rat anti-Calgranulin A clone 8H6, in-house MPIIB (1:100), rabbit anti-histone H3 antibody (citrulline R2+R8+R17; ab5103, Abcam), rabbit anti-histone citrulline H4 antibody (citrulline 3) Millipore 07-596 (1:100) chicken anti-Histone H2B ab134211 Abcam (1:400), rabbit anti-Fibrinogen Millipore ABT259 (1:200). Secondary antibodies were the following: Donkey anti-rabbit immunoglobulin G (IgG) heavy and light chain (H&L) Alexa Fluor 488 (Jackson 711-225-152) and donkey anti-mouse IgG H&L Cy3 (Jackson 715-165-151). Donkey anti-chicken IgY H&L Cy3 (Jackson 703-165-155), donkey anti-rat immunoglobulin G (IgG) heavy and light chain (H&L) Cy5 (Jackson). An upright widefield microscope (Leica DMR) equipped with a JENOPTIK B/W digital microscope camera or a Leica confocal microscope SP8 were used for fluorescent imaging. Z-stack images were collected at 63× magnification. Human tonsil and kidney paraffin tissue block was purchased from AMSbio and paraffin sections of inflamed tissues—gall bladder and appendix—were provided by Dr. Stefan Florian, Charite Berlin.

Cytotoxicity Assay

To assess any toxicity of inhibitors LDH release was assessed using the CytoTox 96® Non-Radioactive Cytotoxicity Assay (Promega), according to the manufacturer's instructions. Neutrophils were seeded in RPMI in a 6 well plate with 1×105 cells/100 μl. Inhibitors were preincubated with cells for 30 min at 37° C. at concentrations indicated in the figures.

Reactive Oxygen Species Assay

Reactive oxygen species (ROS) production was measured using a luminol-HRP assay as described by Amulic et al. (Amulic, B., Knackstedt, S. L., Abu Abed, U., Deigendesch, N., Harbort, C. J., Caffrey, B. E., Brinkmann, V., Heppner, F. L., Hinds, P. W., and Zychlinsky, A. (2017). Cell-Cycle Proteins Control Production of Neutrophil Extracellular Traps. Dev Cell 43, 449-462 e445). Additionally, as all stages of the assay were performed in atmospheric conditions, normal tissue culture media was replaced with a carbonate and phenol red-free RPMI supplemented as before (Seahorse XF RPMI Medium #103336-100, Agilent). Inhibitors were preincubated with cells for 30 min at 37° C. at concentrations indicated in the figures. Immediately before stimulation, luminol (50 μM) and HRP (1.2 U/ml) were added followed by 50 nM PMA. Luminesce measurements were taken every 30 s using a luminescence plate reader (VICTOR Light luminescence counter, Perkin Elmer) and expressed as Relative Light Units (RLU).

Mass Spectrometry

Protein spots were excised and transferred to 0.5 ml Eppendorf tubes. Samples were destained in 500 μl of 200 mM ammoniumbicarbonate (ABC) in 50% acetonitrile (ACN) for 30 min at 37° C. and equilibration in 200 μl of 50 mM ABC, 5% ACN for 30 min at 37° C. The samples were then dried at room temperature for 60 min. Protein digestion was performed overnight at 37° C. with 100 ng trypsin (spots 1, 3, 7 with 200 ng) in 25 μl of 50 mM ABC, 5% ACN. The supernatants were transferred into new Eppendorf tubes and additional peptides were extracted by applying 25 μl of 60% ACN, 0.5% trifluoroacetic acid (TFA) for 10 min, followed by 25 μl of 100% ACN for 10 min. All supernatants were combined and dried in an Eppendorf Concentrator at 45° C. After solubilization in 15 μl of 0.1% TFA, the peptides were desalted with ZipTips and eluted with 1 μl a-cyano-4-hydroxycinnamic acid (5 mg/ml in 60% CAN, 0.3% TFA) onto the MALDI plate. Peptide mass fingerprints (PMF) and fragment spectra (MSMS) of the five most intense peaks were measured using a 4700 Proteomics Analyzer (AB Sciex). The database search was performed applying the Mascot MS/MS Ion Search function for searching the Swiss-Prot subset of human proteins. A peptide mass tolerance of 30 ppm and ±0.3 Da for the fragment mass tolerance was allowed. One missed cleavage, oxidation of methionine, N-terminal acetylation of the protein, propionamide at cysteine residues and N-terminal pyroglutamic acid formation were defined as variable modifications. The following identification criteria were used: minimum 30% sequence coverage; or minimum 15% sequence coverage and one MS/MS confirmation; or sequence coverage below 15% and at least two MS/MS confirmations.

Immunoprecipitation (IP) of Clipped Histones

Magnetic Protein G coupled beads (Invitrogen) were washed (×3) with equal volume PBS. For each IP sample 20 μl of beads were prepared. As 3D9 was a mouse IgG1, a bridging antibody (anti-mouse, Active motif #53017) was used to improve binding to protein G couple beads. Bridging antibody (50 μg) was incubated with 20 μl of magnetic beads for 1 h at 4° C. with gentle inversion. Beads were washed twice with PBS. Beads were then incubated with 50 μg 3D9 or isotype control (IgG1, k—Genscript clone 1H7A8) for 3 h at RT (with gentle rotation). Beads were washed ×3 and resuspended in 2 ml of PBS in a 15 ml falcon tube. To permanently crosslink the antibodies, PFA fixation was used. 2 ml of 1% PFA (in PBS) was added to the beads and vortexed immediately for 1 min. The reaction was then quenched with 125 mM glycine (pH 8.0) and incubated on ice for 5 min. Beads were then washed ×5 with IP lysis buffer (20 mM Tris, 150 mM NaCl, 1% Triton X-100, pH 7.5) and the blocked for 1 h at RT with 1% BSA in IP lysis buffer. Beads were used immediately or stored overnight at 4° C. The next day, neutrophils (1×10⁷) were seeded in Petri dishes with 10 ml of medium, left unstimulated or stimulated with 100 nM PMA for 3 h at 37° C. After stimulation, cells/NETs were washed (×3) very gently with equal volume of PBS (plus 1 mM AEBSF). After the last wash NETs were digested in 1 ml of Benzonase buffer (20 mM Tris-HCl, pH 7.6, 2 mM MgCl₂, 1 mM CaCl₂)) with 250U benzonase (Sigma E1014) and 1 mM AEBSF, 20 μM NEi and CGi for 30 min at 37° C., with intermittent rocking to distribute the buffer. The reaction was stopped with the addition of 4 mM EDTA and 4 mM EGTA. Cells were further lysed with the addition of 300 μl of 5× IP lysis buffer (plus 5× protease inhibitors). Dishes were incubated on ice for 10 min. Cells and NETs were then collected by scraping into an Eppendorf tube. For multiple dishes at the same time point and stimulation, the samples were pooled and divided for the IP step which was performed immediately. For each IP, lysate from 1×10⁷ cells was used with 15 μl of coupled beads and incubated overnight at 4° C. with gentle rotation. The next day beads were washed 5 times with IP lysis buffer plus inhibitors. After the final wash beads were resuspended in 30 μl 1×SDS-PAGE sample loading buffer. Samples were analysed by SDS-PAGE and stained or immunoblotted as indicated. Coomassie Instant blue stain or Thermofisher silver staining kit were used according to the manufacturer's instructions.

Peptide Competition Assay

Neutrophils were seeded on coverslips in a 24 well dish (1×10⁵ cells/well) and stimulated with PMA for 3 h and fixed with 2% PFA. Prior to staining, 3D9 (2 ug/ml=˜12.9 nM) was preincubated overnight with rotation at 4° C. in PBS with 0.01-200× fold molar excess of branched REIRR peptide. TGGVK branched peptide was used as negative control. The next day competed antibody-peptide solutions were diluted 1:1 with blocking buffer and the immunostaining procedure was followed as before. Peptide competition was assessed visually by fluorescent microscopy.

Epitope Mapping

Antibody binding to human H3 was assessed by a combination of linear, conformational and amino acid replacement analysis epitope mapping with peptide synthesis and ELISA assays performed by Pepscan Presto B. V. (Leiden, The Netherlands). To reconstruct epitopes of the target molecule (PATGGVKKPHRYRPGTVALREIRRYQKSTELLIRKLPFQRL H3 amino acid residues 30-70 of SEQ ID NO: 25) a library of peptide-based peptide mimics was synthesized using Fmoc-based solid-phase peptide synthesis. An amino functionalized polypropylene support was obtained by grafting with a proprietary hydrophilic polymer formulation, followed by reaction with t-butyloxycarbonyl-hexamethylenediamine (BocHMDA) using dicyclohexylcarbodiimide (DCC) with N-hydroxybenzotriazole (HOBt) and subsequent cleavage of the Boc-groups using trifluoroacetic acid (TFA). Standard Fmoc-peptide synthesis was used to synthesize peptides on the amino-functionalized solid support by custom modified JANUS liquid handling stations (Perkin Elmer). Synthesis of structural mimics (conformational analysis) was done using Pepscan's proprietary Chemically Linked Peptides on Scaffolds (CLIPS) technology. The binding of antibody to each of the synthesized peptides was tested in a Pepscan-based ELISA. The peptide arrays were incubated with primary antibody (3D9 or isotype control) at 0.02 μg/ml for linear and conformational analysis and 0.5 ug/ml for amino acid replacement analysis (overnight at 4° C.). After washing, the peptide arrays were incubated with a 1/1000 dilution of rabbit anti-mouse IgG(H+L) HRP conjugate (Southern Biotech, Birmingham, AL, USA) for one hour at 25° C. After washing, the peroxidase substrate 2,2′-azino-di-3-ethylbenzthiazoline sulfonate (ABTS) and 20 μl/ml of 3% H₂O₂ were added. After one hour, the colour development was quantified using a charge coupled device (CCD)—camera and an image processing system. The values obtained from the CCD camera ranged from 0 to 3000 mAU, similar to a standard 96-well plate ELISA-reader. The results were quantified and stored in the Peplab database. To verify the quality of the synthesized peptides, a separate set of positive and negative control peptides was synthesized in parallel. These were screened with commercial antibodies 3C9 and 57 (Posthumus, W. P., Lenstra, J. A., Schaaper, W. M., van Nieuwstadt, A. P., Enjuanes, L., and Meloen, R. H. (1990). Analysis and simulation of a neutralizing epitope of transmissible gastroenteritis virus. J Virol 64, 3304-3309).

Hybridoma Antibody Gene Sequencing

Sequencing of the antibody gene of hybridoma 3D9 was performed by Genscript (NJ, USA). Briefly, total RNA was isolated from 5 clones of the 3D9 hybridoma following the technical manual of TRIzol® Reagent. Total RNA was then reverse-transcribed into cDNA using either isotype-specific anti-sense primers or universal primers following the technical manual of PrimeScript™ 1st Strand cDNA Synthesis Kit. Antibody fragments of heavy chain and light chain were amplified according to Genscript's standard operating procedure for rapid amplification of cDNA ends (RACE). Amplified antibody fragments were cloned into the cloning vector pCE2 TA/Blunt-zero Vector (CAT #C601-01-Vazyme Biotech Co., Ltd, China). Colony PCR was performed to screen for clones with inserts of correct sizes. The consensus sequence was provided and the fragments sequenced using an Applied Biosystems 3730 DNA Analyzer. Sequences were then aligned and annotated using the results of NCBI IgBlast.

TABLE 2
Description Sequence Listing

| SEQ ID NO | Description | Type |
|---|---|---|
| 1 | 3D9 Heavy chain | Amino acid |
| 2 | 3D9 Light chain | Amino acid |
| 3 | H-CDR1 | Amino acid |
| 4 | H-CDR2 | Amino acid |
| 5 | H-CDR3 | Amino acid |
| 6 | L-CDR1 | Amino acid |
| 7 | L-CDR2 | Amino acid |
| 8 | L-CDR3 | Amino acid |
| 9 | 3D9 Heavy chain | Nucleic acid |
| 10 | 3D9 Light chain | Nucleic acid |
| 11 | Peptide used for immunization linked | Amino acid |
| | via amide linkage between the carboxyl | |
| | end of Arg and the side chain amine of | |
| | the Lys of SEQ ID NO 12 or of SEQ ID | |
| | NO 15 or of SEQ ID NO 26 | |
| 12 | Peptide used for immunization with | Amino acid |
| | SEQ ID NO 11 and conjugated to KLH | |
| 13 | Peptide used for negative screening | Amino acid |
| | linked via amide linkage between the | |
| | carboxyl end of Ser and the side chain | |
| | amine of Lys6 of SEQ ID NO 14 | |
| 14 | Peptide used for negative screening with | Amino acid |
| | SEQ Id NO 13 | |
| 15 | Competing peptide used for validation | Amino acid |
| | with SEQ ID NO 11 | |
| 16 | Negative control peptide used for | Amino acid |
| | validation linked via amide linkage | |
| | between the carboxyl end of Lys and | |
| | the side chain amine of Lys6 of SEQ | |
| | ID NO 17 | |
| 17 | Negative control peptide used for | Amino acid |
| | validation with SEQ ID NO 16 | |
| 18 | Intact H3 | Amino acid |
| 19 | CS2 round 1 | Amino acid |
| 20 | CS2 round 2 | Amino acid |
| 21 | CS2 round 2 | Amino acid |
| 22 | CS3 round 1 | Amino acid |
| 23 | CS3 round 2 | Amino acid |
| 24 | CS4 round 1 | Amino acid |
| 25 | Human histone H3 | Amino acid |
| 26 | Peptide used for positive screening | Amino acid |
| | with SEQ ID NO 11 | |

### Example 1: Serine Protease Dependent Cleavage of Histone H3 N-Terminal Tails During NET Formation

Histones are processed in response to phorbol 12 myristate 13 acetate—PMA and other NET stimuli. In a time course experiment of human neutrophils incubated with PMA, the inventors detected a H3 cleavage product of ˜14 kDa as early as 30 min post-stimulation (FIG. 1), later yielding further cleavage products of approximately 13 kDa and 10 kDa. A C-terminal, but not an N-terminal, histone antibody detected the cleavage products of H3 (FIG. 1A). FIG. 1A shows that PMA induced Histone H3 cleavage occurs in the N-terminal domain and is prevented by serine protease inhibition. These results indicate that the N-terminus is clipped in truncated H3.

Neutrophil azurophilic granules are rich in serine proteases that can degrade histones in vitro. Thus, the contribution of these proteases to H3 cleavage during NET formation was tested. Preincubation with the serine protease inhibitor, AEBSF (4-[2-aminoethyl] benzensulfonylfluoride) (FIG. 1A) inhibited H3 cleavage upon PMA stimulation. AEBSF also inhibited NET formation and nuclear decondensation as shown by immunofluorescent microscopy (FIG. 1B). This data shows that serine proteases cleave the N-terminus of H3 early during NET formation.

### Example 2: Histone H3 is Cleaved at a Novel Site in the Globular Domain

To identify the precise H3 cleavage sites the inventors prepared histone enriched extracts from primary neutrophils stimulated with PMA for 90 min and then purified H3 as described in the Method section. The inventors identified the fractions containing H3 and its cleaved products by western blot with anti-H3 C-terminal antibodies. H3 was the last core histone to elute at 45-46 min (FIG. 2A). The inventors separated H3 and its truncated forms by 2D-electrophoresis (2D-E) and confirmed their identity by mass spectrometry (FIG. 2A). FIG. 2B shows representative Coomassie stained blot of pooled H3 fractions separated by 2D-electrophoresis.

The sequence coverage did not include residues that allowed differentiation of H3 variants. The N-terminals of the separated H3 fragments shown in FIG. 2B were sequenced by Edman degradation from 2 independent experiments (Table 1). The N-terminal sequence of the largest molecular weight H3 spot matched that of intact H3 (spot 1). We did not obtain reliable sequencing of spots 2 and 3 but the cleavage sites of spots 4 and 5 were identified. The most truncated H3 fragment (spot 5) was cleaved between L48 and R49, in the globular domain of the protein, within the nucleosome core structure. This is a previously unidentified cleavage site in H3 and, thus, the inventors selected cleavage at H3R49 as a candidate marker of NETs.

TABLE 1
N-terminal histone H3 cleavage sites identified
by Edman degradation sequencing. In the second
experiment overlapping sequences were detected.
However, the detected amino acids for spot 4
and 5 confirmed the initial sequence
identification.

| Spot # | Name | Round 1 | Round 2 |
|---|---|---|---|
| 1 | Intact | ARTKQ | ARTKQ |
| | H3 | (SEQ ID NO 18) | (SEQ ID NO 18) |
| 2 | CS1 | not analysable | T |
| 3 | CS2 | AARKS | AAAAS |
| | | (SEQ ID NO 19) | (SEQ ID NO 20) |
| | | | TKRRR |
| | | | (SEQ ID NO 21) |
| 4 | CS3 | TGGV | TGGVK |
| | | (SEQ ID NO 22) | (SEQ ID NO 23) |
| | | | AAA |
| 5 | CS4 | REIRR | REIRR |
| | | (SEQ ID NO 24) | (SEQ ID NO 24) |
| | | | AL (residues 1, 2), |
| | | | EI (residues 4, 5) |

### Example 3: Generation of a Histone H3 Cleavage Site Monoclonal Antibody

In order to raise an antibody against the cleaved histone site, the inventors immunized mice with a lysine branched immunogen containing the 5 amino acids carboxyl to the H3R49 cleavage site (FIG. 3D). First, sera were screened—and later the hybridoma supernatants—against the immunogen (branched peptide formed by SEQ ID NO 11 and 12) and control peptides (branched peptide formed by SEQ ID NO 13 and 14) by ELISA. Then a screen was performed for sera or clones that detected cleaved H3 in immunoblots of PMA stimulated neutrophil cell lysates. Sera and clones were excluded that detected full length histone H3 in addition to cleaved H3 (FIGS. 3A and 3B). In parallel, sera were screened to select clones that detected NETs but not resting chromatin of naïve neutrophils by immunofluorescence microscopy. Of the 6 mice immunised, one stable clone was obtained, 3D9, that functioned in both western blot and microscopy.

3D9 recognised a protein of ˜10 kDa in neutrophils stimulated with PMA for 120 min and longer but did not detect any protein in resting or early stimulated cells (FIG. 3B). This band corresponded in size with the smallest H3 fragment detected by the H3 C-terminal antibody. Interestingly, by microscopy 3D9 exclusively recognised neutrophils undergoing NETosis— with decondensing chromatin (FIG. 3A). To validate the specificity of the antibody for the de novo N-terminal H3 epitope of NETs, the inventors performed competition experiments with the branched immunising peptide (SEQ ID NO 11 and 12) and the branched negative control peptide (SEQ ID NO 16 and 17) and demonstrated that it could block 3D9 binding to NETs as shown by immunofluorescent microscopy (FIG. 3E).

Indirect Elisa experiment showed that 3D9 binds specifically to cleaved H3, as 3D9 bound to the branched immunizing peptide comprising sequence REIRR (SEQ ID NO 11 and 24) and to isolated NETs, but not to equal concentrations of chromatin, recombinant H3 or purified calf thymus DNA, by direct ELISA (FIG. 3C). Furthermore, in immunoprecipitation experiments, 3D9, but not an isotype control, pulled down H3 both in lysates of naïve and activated neutrophils, but only the cleaved fragment from activated cells (FIG. 3F). The inventors detected these pull downs both by Coomassie and silver stained gels. In a similar experiment, the immunoprecipitate was immunoblotted with the C-terminal antibody as well as 3D9 (FIG. 3G), and showed that only the inventive monoclonal recognizes cleaved H3. Consequently, these data show that 3D9 is selective for cleaved H3 under the denaturing conditions of SDS-PAGE but also recognizes full length H3 under the more native conditions of IP.

### Example 4: Epitope Mapping of Antibody Clone 3D9 to Histone H3

The inventors determined the binding site of 3D9 in histone H3 by ELISA with overlapping linear peptide arrays and helical peptide mimic arrays, based on a sequence (PATGGVKKPHRYRPGTVALREIRRYQKSTELLIRKLPFQRL=residues 30-70 of SEQ ID 25) around the H3R49 cleavage site. To mimic potential charges created at the newly revealed N terminus, R49, arrays were included of unmodified peptides. Based on overlapping peptides, the putative core epitope in the linear array was (R)EIRR. The peptides ending in REIRR were in all cases in the top 2 of each peptide mimic. Interestingly, peptides extended at the N-terminus were still recognized. Moreover, acetylation at the N-terminus of the peptide ending in the REIRR sequence did not affect the binding, suggesting that a free N-terminus may not be recognized by the antibody. The epitope mapping was refined by amino acid replacement analysis of linear peptides and helical peptide mimetics ending in REIRR. Mutations in Glu51, Ile52, and Arg54 negatively impacted the signal, indicating these residues are critical for epitope recognition.

### Example 5: Automatic Quantification of In Vitro Generated NETs by Microscopy

The inventors tested how 3D9 stained NETs by immunofluorescence microscopy in samples that were permeabilized (Triton X-100, 0.5% for 10 min) to facilitate the distribution of the antibody throughout the sample. 3D9 detected almost exclusively decondensed chromatin (FIG. 4A). In contrast, the anti-chromatin antibody (clone PL2.3)—directed against a H2A-H2B-DNA epitope—stained NETs in addition to condensed nuclei.

The staining characteristics of 3D9 and PL2.3 were compared during NET formation. Nuclear area and signal intensity were determined at the indicated time points, from multiple fields of view (FIG. 4B). Both antibodies detected the increase in nuclear area characteristic of NETosis between 15 and 180 min after simulation. At later time points, the intensity of PL2.3 staining decreased and failed to discriminate between resting cell nuclei and NETs. In contrast, 3D9 stained nuclei undergoing NETosis more brightly than nuclei of non-activated cells.

Publicly available software ImageJ can be used to quantify in vitro NETosis. The staining with 3D9 was compared versus the anti-chromatin antibody by a previously published manual semi-automatic image analysis (Brinkmann et al., 2012) and a modified automatic method. Both methods use automatic thresholding of the DNA channel (Hoechst) to count total cells/objects. The historical manual semi-automatic method exploits the differential staining by chromatin antibodies of decondensed chromatin (high signal) over compact chromatin (weak signal) to count cells in NETosis. This method uses a manual thresholding and segmentation procedure (denoted manual in the figure). This manual thresholding step is subject to observer bias. In contrast, the modified method uses automatic thresholding at both stages; total cell and NET counts. Both methods use a size exclusion particle analysis step so that only structures larger than a resting nucleus are counted. Both 3D9 and PL2.3 antibodies effectively quantified NETs using previously published method (FIG. 4C—manual). However, PL2.3 failed to accurately quantify the number of NETs with the fully automatic method, specifically at early time points after stimulation (15 min) where the weakly staining lobulated nucleus can extend over a larger surface area during cell activation and adhesion. Together, these data indicate that the modified automatic method using 3D9 staining reduces experimental bias.

### Example 5: 3D9 Detects NETs Induced by Multiple Stimuli

Histone H3 cleavage happens in NETs induced by multiple stimuli. 3D9 detected NETs induced by the bacterial toxin nigericin, which induces NETs independently of NADPH oxidase activation, by heme in TNF primed neutrophils and by the fungal pathogen Candida albicans (FIG. 5). Interestingly, in C. albicans infections, both 3D9 positive and negative NETs were detected.

### Example 6: 3D9 Distinguishes NETs in Mixed Cell Samples

Histone H3 clipping, albeit at other sites in the N-terminal tail, was observed in mast cells and unstimulated PBMC fractions. Of note, PBMC fractions often contain contaminating neutrophils. To test if 3D9 specifically stained neutrophils treated with NET stimuli, PBMCs were incubated with PMA or nigericin (FIG. 6). Importantly, 3D9 detected only nuclei that appeared decondensed in cells that were positive for NE, a specific neutrophil marker. In contrast, the chromatin antibody stained both neutrophils in NETosis and nuclei of other cells. This shows that 3D9 detects NETs specifically even in the presence of other blood cells.

### Example 7: 3D9 Distinguishes NETosis from Other Forms of Cell Death in Neutrophils

It is well known that neutrophils can commit to different cell death pathways. The inventors induced apoptosis by overnight incubation and necroptosis with TNFα stimulation in the presence of a SMAC (second mitochondria-derived activator of caspase) mimetic and caspase inhibition. Interestingly, the anti-chromatin antibody PL2.3 (FIG. 7B), but not 3D9 (FIG. 7A), stained the condensed nuclei of cells undergoing apoptosis and neither of the antibodies stained cells during necrosis induced by the staphylococcal toxin α-haemolysin.

### Example 8: 3D9 Labels NETs in Human Tissue Sections

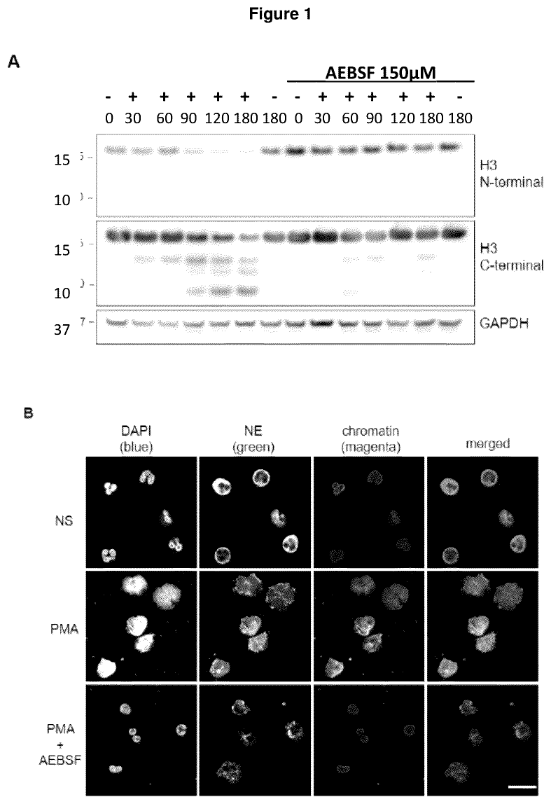

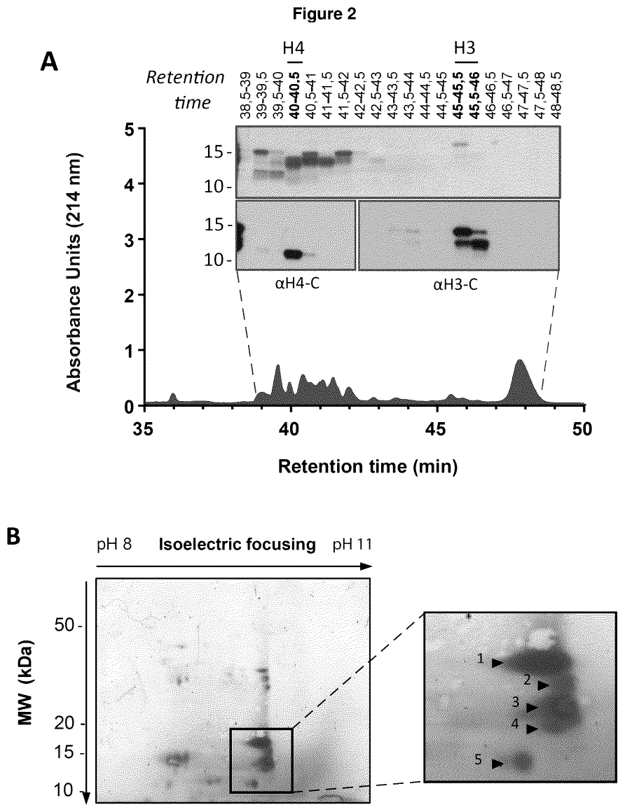

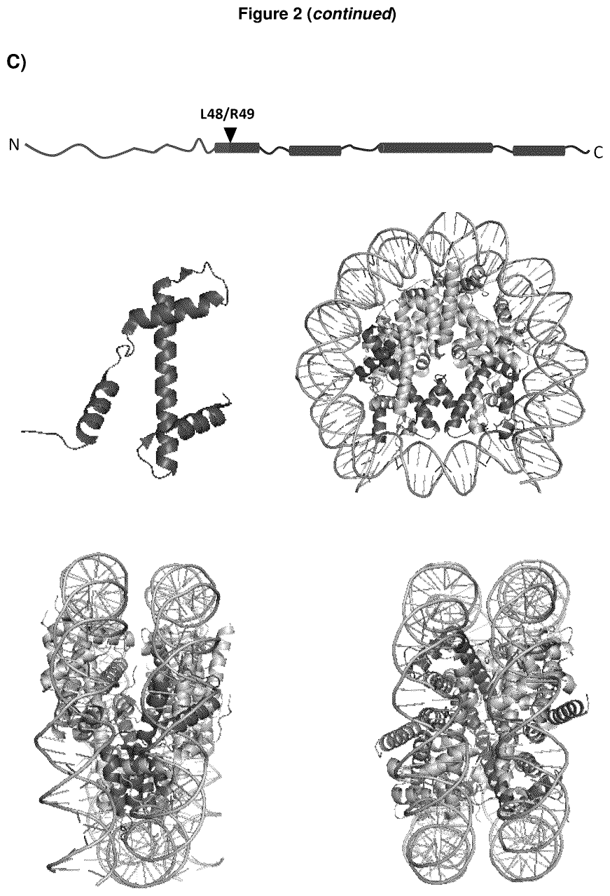

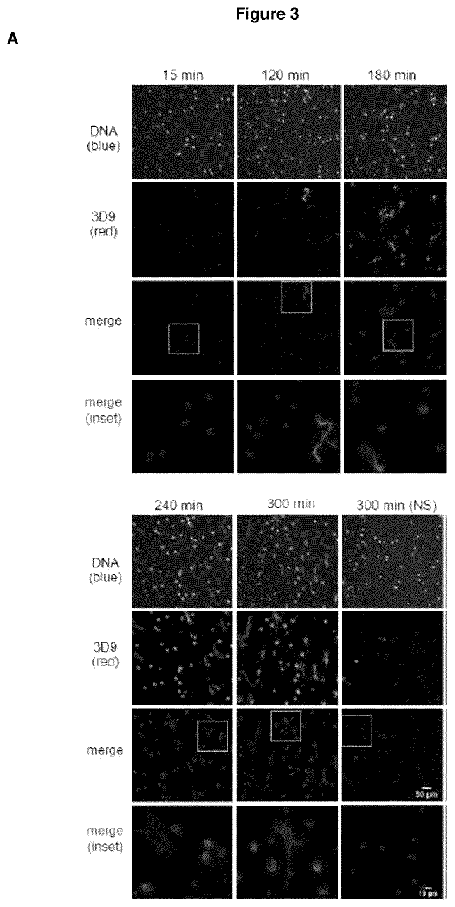

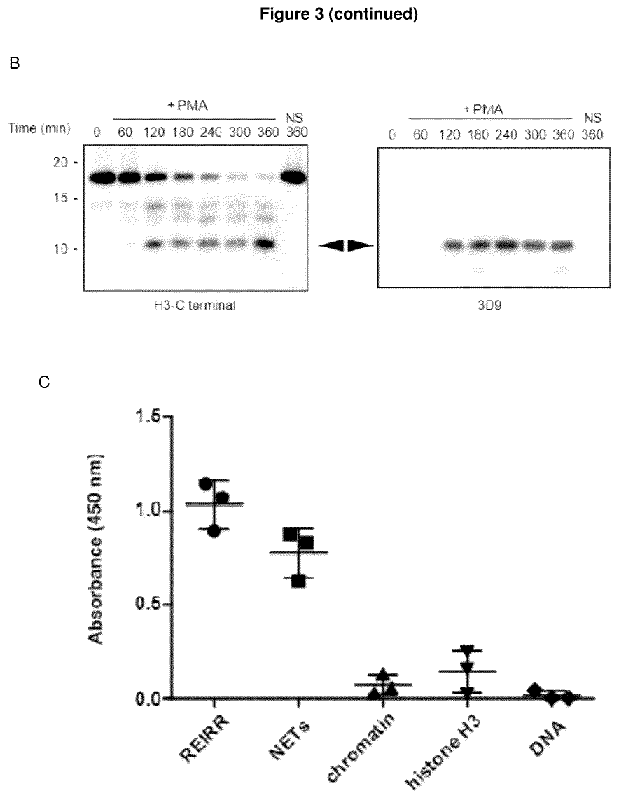

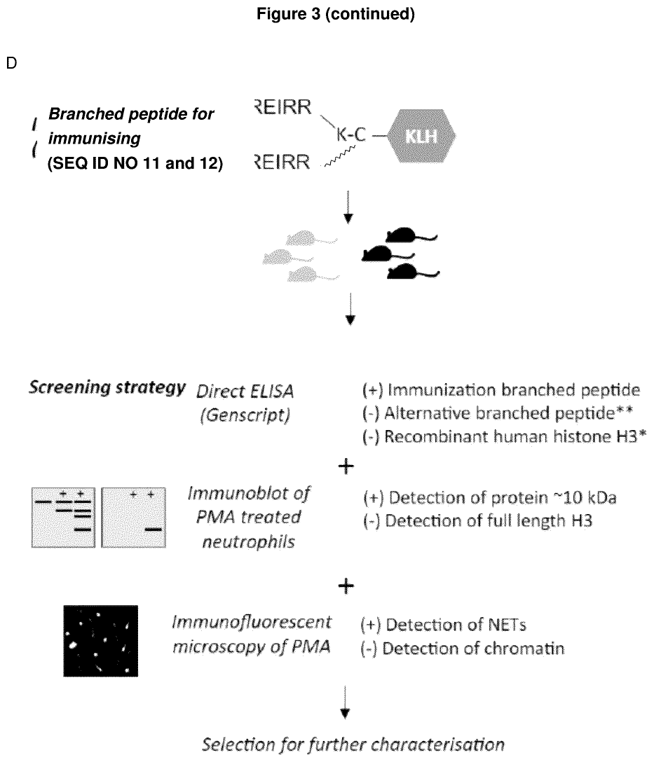

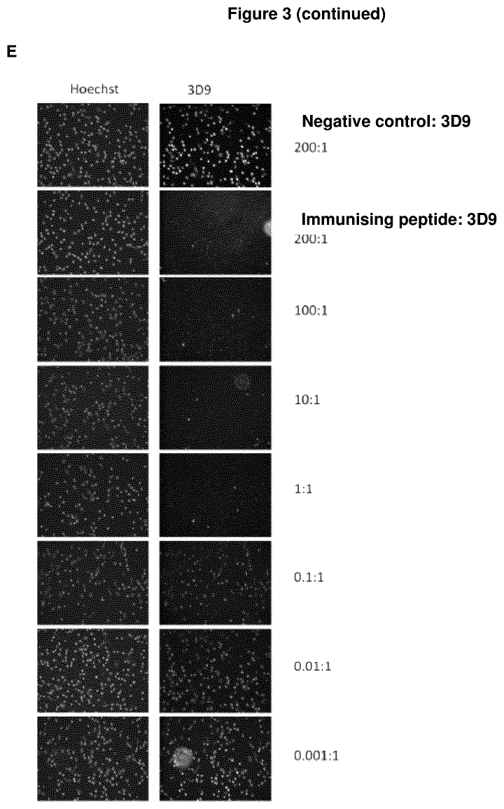

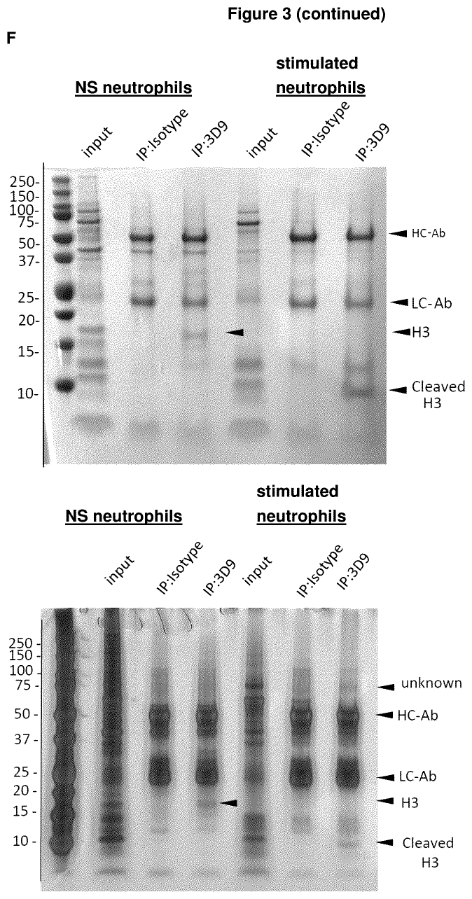

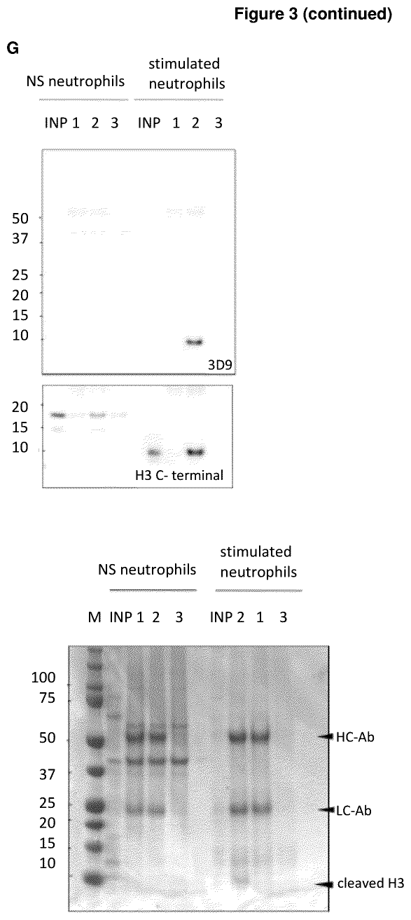

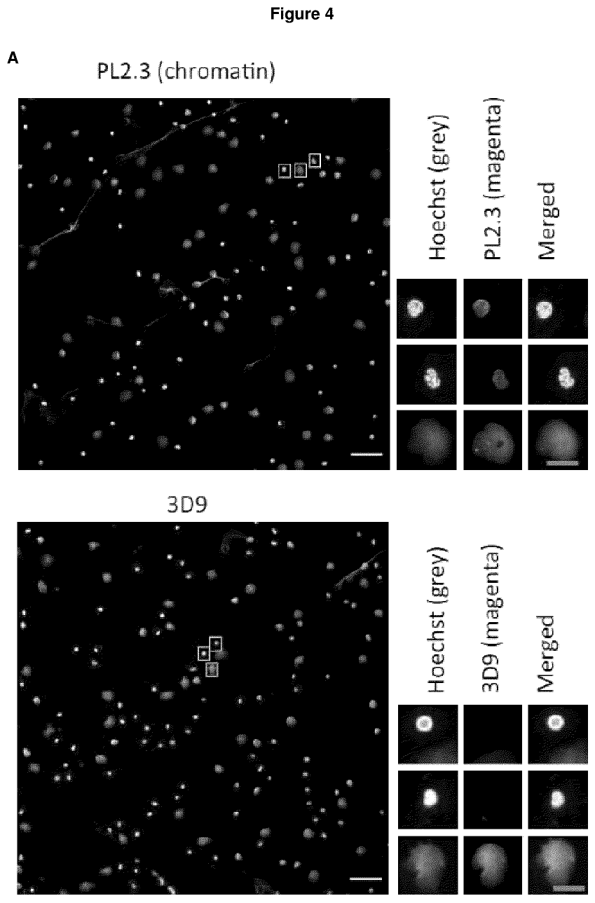

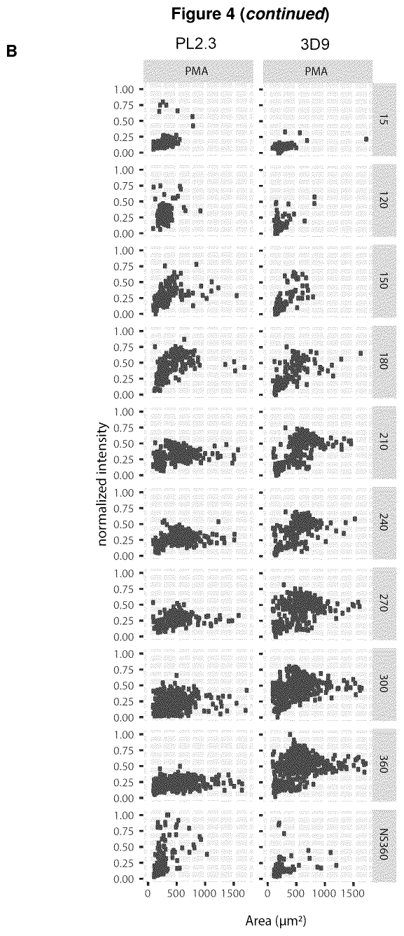

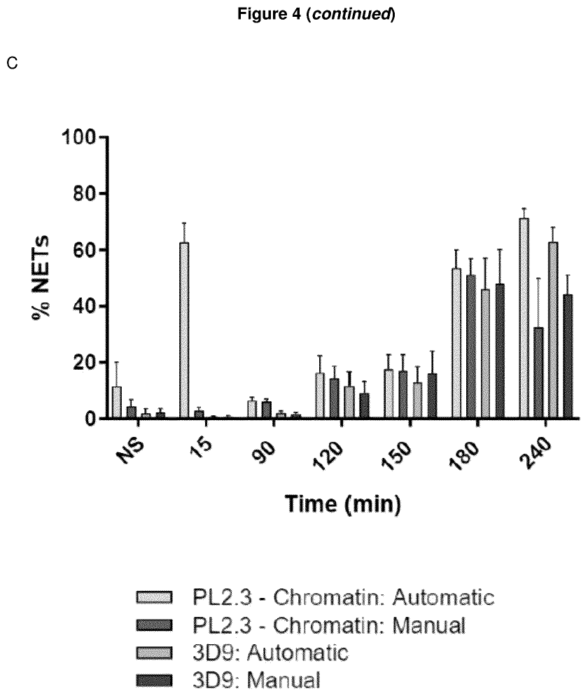

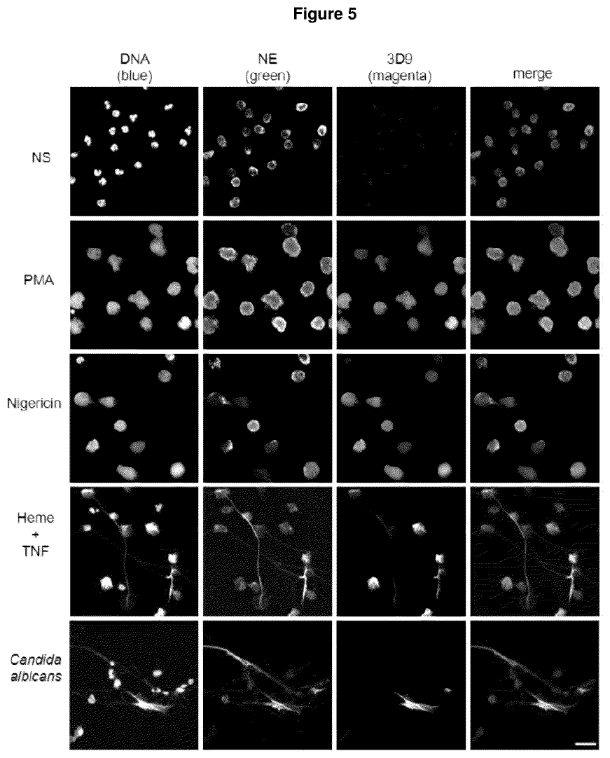

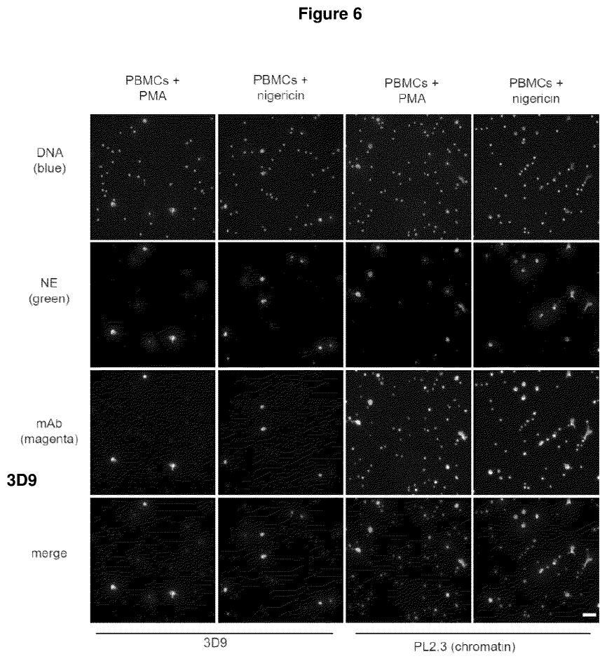

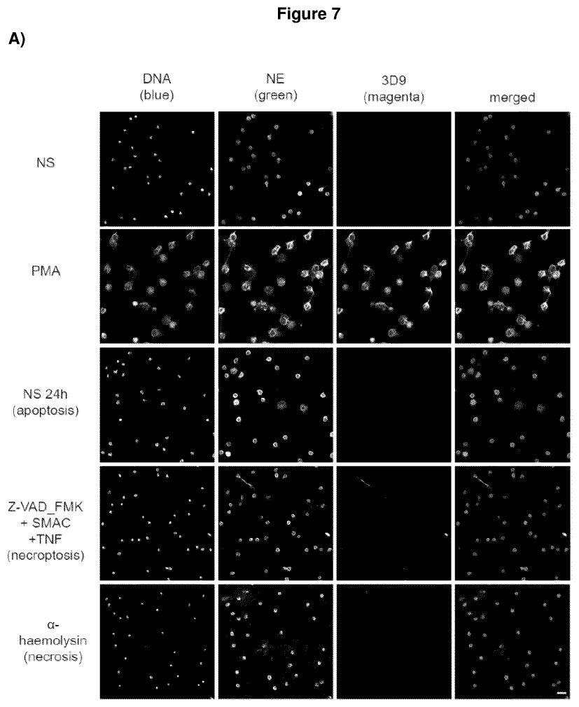

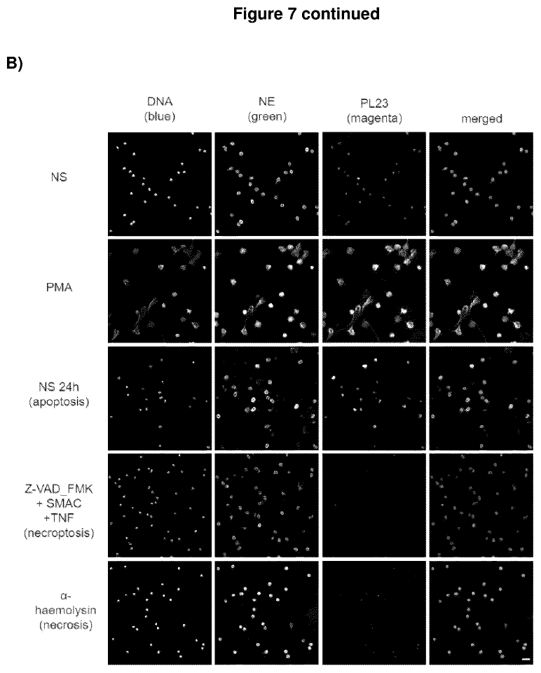

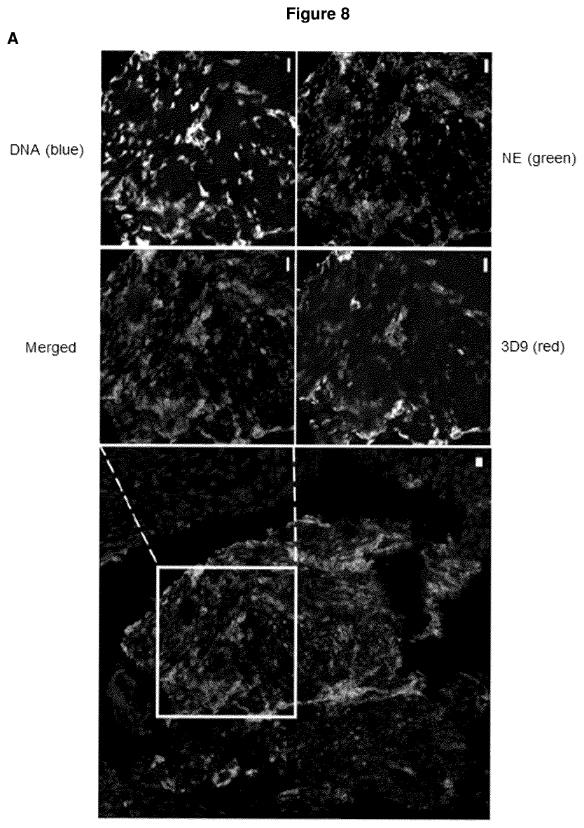

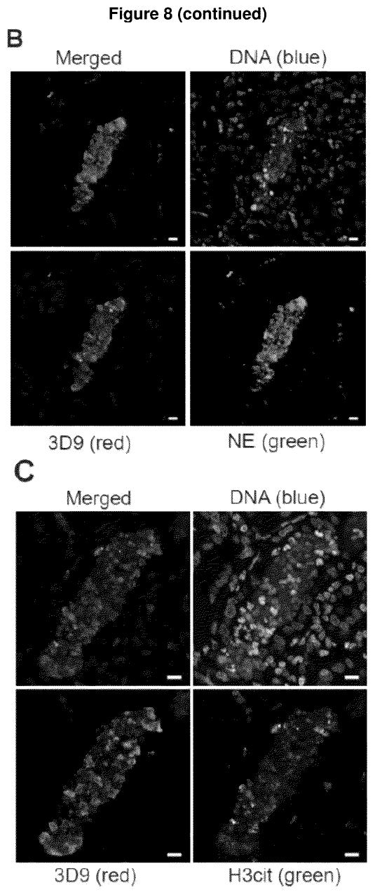

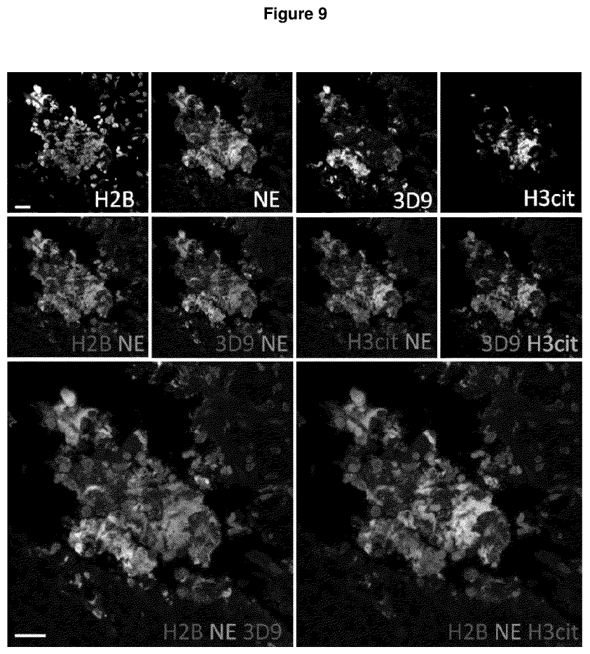

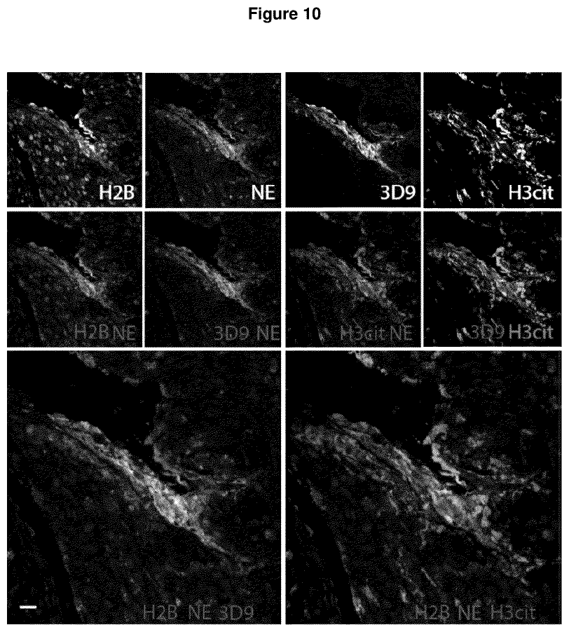

NETs are found in inflamed tissues based on the juxtaposition of chromatin and granular makers as well as the detection of citrullinated H3. 3D9 stains areas of decondensed DNA (Hoechst) that colocalise with NE in both inflammed human tonsil (FIG. 8A), human kidney (FIG. 8B). This indicates that 3D9 labels NETs in histological samples. Indeed, in kidney (FIG. 8C), inflamed appendix (FIG. 9), and gall bladder (FIG. 10), 3D9 labelled DNA in the same cluster as anti-H3cit or anti-H2B antibodies. Interestingly, 3D9 stained decondensed, more NET-like structures, while anti-H3cit or anti-H2B antibodies stained relatively compact chromatin.

### Example 9: Identification of Cleavage Site L48R49 on Other Histone H3 Variants

By performing sequence alignment analysis, it has been observed that the cleavage site at L48R49 of SEQ ID NO 25, which corresponds to Histone H3 sequence Q71 DI3 without the first Met, is present at the same position also in the other histone H3 variants P0DPK2, Q6NXT2, Q16695, P84243, and P68431. Thus, the cleavage site L48R49 of SEQ ID NO 25 corresponds to the site L49R50 of Q71 DI3, P0DPK2, Q6NXT2, Q16695, P84243, and P68431.

What is claimed is:

1. An isolated antibody specifically binding a C-terminal fragment of cleaved histone H3 or an antigen-binding portion thereof, wherein the antibody comprises:
a) H-CDR1 comprising an amino acid sequence of SEQ ID NO: 3, or an amino acid sequence having a sequence identity of more than 90% with of SEQ ID NO: 3;
b) H-CDR2 comprising an amino acid sequence of SEQ ID NO: 4, or an amino acid sequence having a sequence identity of more than 90% with of SEQ ID NO: 4;
c) H-CDR3 comprising an amino acid sequence of SEQ ID NO: 5, or an amino acid sequence having a sequence identity of more than 90% with of SEQ ID NO: 5;
d) L-CDR1 comprising an amino acid sequence of SEQ ID NO: 6, or an amino acid sequence having a sequence identity of more than 90% with of SEQ ID NO: 6;
e) L-CDR2 comprising an amino acid sequence of SEQ ID NO: 7, or an amino acid sequence having a sequence identity of more than 90% with of SEQ ID NO: 7;
f) L-CDR3 comprising an amino acid sequence of SEQ ID NO: 8, or an amino acid sequence having a sequence identity of more than 90% with of SEQ ID NO: 8.

2. The isolated antibody specifically binding a C-terminal fragment of cleaved histone H3 or an antigen-binding portion thereof according to claim 1, wherein the antibody comprises:
I) a heavy chain variable region comprising an amino acid sequence of SEQ ID NO: 1, or an amino acid sequence having a sequence identity of more than 90% with of SEQ ID NO: 1; and
II) a light chain variable region comprising an amino acid sequence of SEQ ID NO: 2, or an amino acid sequence having a sequence identity of more than 90% with of SEQ ID NO: 2.

3. The isolated antibody specifically binding a C-terminal fragment of cleaved histone H3 or an antigen-binding portion thereof according to claim 1, wherein the antigen binding fragment is selected from the group comprising single chain variable fragments (scFv), variable domain (Fv) fragments, fragment antigen binding (Fab) fragments, F(ab)2 fragments, peptides, or proteolytic fragments containing an epitope binding region.

4. A nucleic acid composition comprising one or more nucleic acid molecules encoding an antibody specifically binding a C-terminal fragment of cleaved histone H3 or an antigen-binding portion thereof according to claim 1.

5. A nucleic acid composition comprising one or more nucleic acid molecules encoding an antibody specifically binding a C-terminal fragment of cleaved histone H3 or an antigen-binding portion thereof according to claim 1, wherein the nucleic acid composition comprises a nucleic acid sequence having a sequence identity of more than 90% with SEQ ID NO: 9, and a nucleic acid sequence having a sequence identity of more than 90% with SEQ ID NO: 10.

6. A host cell comprising the recombinant nucleic acid molecule according to claim 4.

7. A method for in vitro detection of neutrophil extracellular traps in an isolated biological sample, the method comprising:
1) contacting said isolated biological sample with an antibody specific for a C-terminal fragment of cleaved histone H3 or an antigen-binding portion thereof according to claim 1,
2) providing conditions sufficient to allow said antibody specific for a C-terminal fragment of cleaved histone H3 or an antigen-binding portion thereof to bind to said isolated biological sample,
3) determining whether the antibody specific for a C-terminal fragment of cleaved histone H3 or an antigen-binding portion thereof binds to said isolated biological sample.

8. The method according to claim 7, wherein the antibody specific for a C-terminal fragment of cleaved histone H3 or an antigen-binding portion thereof is linked to a detectable label.

9. A method for in vitro assessing a disease condition in an individual:
i) contacting an isolated biological sample obtained from said individual with an antibody specific for a C-terminal fragment of cleaved histone H3 or an antigen-binding portion thereof according to claim 1;
ii) providing conditions sufficient to allow said antibody specific for a C-terminal fragment of cleaved histone H3 or an antigen-binding portion thereof to bind to said isolated biological sample,
iii) determining whether the antibody specific for a C-terminal fragment of cleaved histone H3 or an antigen-binding portion thereof binds to said isolated biological sample,
wherein said disease is associated with NET formation,
wherein said disease is selected from the group comprising thrombotic disease, neurological disease, bacterial infection disease, viral infection disease, fungal infection disease, inflammatory disease, autoimmune disease, cancer disease, cancer metastasis disease.

10. The method according to claim 9, wherein the autoimmune disease is selected from the group comprising psoriasis; vasculitis; systemic lupus erythematosus (SLE); rheumatoid arthritis; ulcerative colitis; Crohn's disease; immune-mediated or Type 1 Diabetes Mellitus; immune mediated glomerulonephritis; inflammatory bowel diseases (IBD); antiphospholipid antibody syndrome; immune thrombocytopenia.

11. The method according to claim 9, wherein the antibody specific for a C-terminal fragment of cleaved histone H3 or an antigen-binding portion thereof is linked to a detectable label.

12. An isolated fragment of human histone H3 cleaved at site L48R49 of SEQ ID NO 25, wherein the fragment of human histone H3 cleaved at site L48R49 consists of an amino acid sequence comprised between residues R49-A135 of SEQ ID NO 25, or a segment thereof or consists of an amino acid sequence comprised between residues A1-L48 of SEQ ID NO 25, or a segment thereof.

13. A kit for assessing a disease containing the antibody specific for a C-terminal fragment or an antigen binding portion thereof according to claim 1, wherein the disease selected from the group comprising thrombotic disease, neurological disease, bacterial infection disease, viral infection disease, fungal infection disease, inflammatory disease, autoimmune disease, cancer disease, cancer metastasis disease, and wherein said disease is associated with NET formation.

14. The kit for assessing a disease according to claim 13, wherein the autoimmune disease is selected from the group comprising psoriasis; vasculitis; systemic lupus erythematosus (SLE); rheumatoid arthritis; ulcerative colitis; Crohn's disease; immune-mediated or Type 1 Diabetes Mellitus; immune mediated glomerulonephritis; inflammatory bowel diseases (IBD); antiphospholipid antibody syndrome; immune thrombocytopenia.

15. (canceled)


SEQUENCE LISTING (26 sequences)

SEQ ID NO: 1 - protein, 468 aa, Homo sapiens
MGWICIIFFLVATATGVHSQVQLQQSGPELVRPGVSVKISCKGSGYTFTDYSMHWVRQSHAKSLEWIGIISTYSGNTIYSQNFKGKATMTVDRSSNTAYMEFARLTSEDSAIYYCARGGYDDYDESHYYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 2 - protein, 238 aa, Homo sapiens
MSPAQFLFLLLLWIRETNGDVVMTQTPLTLSVNIGQPASISCKSGQGLLDSDGKTYLNWLLQRPGQSPKRLVFLVSQLDSGVPDRFTGSGSGTDFTLKISRVEAEDLGVYYCWQATHFPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3 - protein, 5 aa, Homo sapiens
DYSMH

SEQ ID NO: 4 - protein, 17 aa, Homo sapiens
IISTYSGNTIYSQNFKG

SEQ ID NO: 5 - protein, 16 aa, Homo sapiens
GGYDDYDESHYYAMDY

SEQ ID NO: 6 - protein, 16 aa, Homo sapiens
KSGQGLLDSDGKTYLN

SEQ ID NO: 7 - protein, 7 aa, Homo sapiens
LVSQLDS

SEQ ID NO: 8 - protein, 9 aa, Homo sapiens
WQATHFPYT

SEQ ID NO: 9 - DNA, 1407 bp, Homo sapiens
atgggttggatctgtatcatcttctttctggtagcaacagctacaggtgtgcactcccaggtccagctccagcagtctgggcctgaactggtgaggcctggggtctcagtgaagatttcctgcaagggttccggctacacattcactgattattctatgcactgggtgaggcagagtcatgcaaagagtctagagtggattggaattattagtacttactctggtaatacaatctacagccagaactttaagggcaaggccacaatgactgttgacagatcctccaacacagcctatatggagtttgccagattgacttctgaggattctgccatctattactgtgcaagagggggctacgatgattacgacgagtcacattactatgctatggactactggggtcaaggaacctcggtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 10 - DNA, 717 bp, Homo sapiens
atgagtcctgcccagttcctgtttctgttactgctctggattcgggaaaccaacggtgatgttgtgatgacccagactccactcactttgtcggttaatattggacaaccagcctccatctcttgcaagtcaggtcagggcctcttagatagtgatggaaagacatatttgaattggttgttacagaggccaggccagtctccaaagcgcctagtctttctggtgtctcaactggactctggagtccctgacaggttcactggcagtggatcagggacagatttcacactgaaaatcagcagagtggaggctgaggatttgggagtttattattgctggcaagcgacacattttccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 11 - protein, 5 aa, Homo sapiens
  SITE (1)..(5): This sequence is one part of a branched amino acid sequence
  SITE (5)..(5): C-terminal amino acid is linked to the side chain amine of the lysine in position 6 of SEQ ID NO 12, or 15, or 26
REIRR

SEQ ID NO: 12 - protein, 7 aa, Homo sapiens
  SITE (1)..(7): This sequence is one part of a branched amino acid sequence
  SITE (6)..(6): the side chain amine of the lysine in position 6 is linked via an amide linkage to the C-terminal amino acid of SEQ ID NO 11
  MOD_RES (7)..(7): AMIDATION
  SITE (7)..(7): this peptide sequence is conjugated to carrier protein key lymphocyte haemoglutinin (KLH) via the thiol group of the cystein in position 7
REIRRKC

SEQ ID NO: 13 - protein, 5 aa, Homo sapiens
  SITE (1)..(5): This sequence is one part of a branched amino acid sequence
  SITE (5)..(5): C-terminal amino acid is linked to the side chain amine of the lysine in position 6 of SEQ ID NO 14
AARKS

SEQ ID NO: 14 - protein, 7 aa, Homo sapiens
  SITE (1)..(7): This sequence is one part of a branched amino acid sequence
  SITE (6)..(6): the side chain amine of the lysine in position 6 is linked via an amide linkage to the C-terminal amino acid of SEQ ID NO 13
  MOD_RES (7)..(7): AMIDATION
AARKSKC

SEQ ID NO: 15 - protein, 6 aa, Homo sapiens
  SITE (1)..(6): This sequence is one part of a branched amino acid sequence
  MOD_RES (6)..(6): AMIDATION
  SITE (6)..(6): the side chain amine of the lysine in position 6 is linked via an amide linkage to the C-terminal amino acid of SEQ ID NO 11
REIRRK

SEQ ID NO: 16 - protein, 5 aa, Homo sapiens
  SITE (1)..(5): This sequence is one part of a branched amino acid sequence
  SITE (5)..(5): C-terminal amino acid is linked to the side chain amine of the lysine in position 6 of SEQ ID NO 17
TGGVK

SEQ ID NO: 17 - protein, 6 aa, Homo sapiens
  SITE (1)..(6): This sequence is one part of a branched amino acid sequence
  MOD_RES (6)..(6): AMIDATION
  SITE (6)..(6): the side chain amine of the lysine in position 6 is linked via an amide linkage to the C-terminal amino acid of SEQ ID NO 16
TGGVKK

SEQ ID NO: 18 - protein, 5 aa, Homo sapiens
ARTKQ

SEQ ID NO: 19 - protein, 5 aa, Homo sapiens
AARKS

SEQ ID NO: 20 - protein, 5 aa, Homo sapiens
AAAAS

SEQ ID NO: 21 - protein, 5 aa, Homo sapiens
TKRRR

SEQ ID NO: 22 - protein, 4 aa, Homo sapiens
TGGV

SEQ ID NO: 23 - protein, 5 aa, Homo sapiens
TGGVK

SEQ ID NO: 24 - protein, 5 aa, Homo sapiens
REIRR

SEQ ID NO: 25 - protein, 135 aa, Homo sapiens
ARTKQTARKSTGGKAPRKQLATKAARKSAPATGGVKKPHRYRPGTVALREIRRYQKSTELLIRKLPFQRLVREIAQDFKTDLRFQSSAVMALQEASEAYLVGLFEDTNLCAIHAKRVTIMPKDIQLARRIRGERA

SEQ ID NO: 26 - protein, 7 aa, Homo sapiens
  SITE (1)..(7): This sequence is one part of a branched amino acid sequence
  SITE (6)..(6): the side chain amine of the lysine in position 6 is linked via an amide linkage to the C-terminal amino acid of SEQ ID NO 11
  MOD_RES (7)..(7): AMIDATION
REIRRKC